(19) United States
(12) Patent Application Publication
(10) Pub. No.: US 2004/0121015 A1
(43) Pub. Date: Jun. 24, 2004

(54) Controlled-Release of an active substance into a high fat environment

(75) Inventors: Mark B. Chidlaw, Bend, OR (US); Dwayne T. Friesen, Bend, OR (US); Scott M. Herbig, East Lyme, CT (US); James A.S. Nightingale, Bend, OR (US); Cynthia A. Oksanen, Stonington, CT (US); James B. West, Bend, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/727,217

(22) Filed: Dec. 3, 2003

Related U.S. Application Data
(60) Provisional application No. 60/432,860, filed on Dec. 11, 2002.

(51) Int. Cl. A61K 9/24

(52) U.S. Cl. 424/471

(57) ABSTRACT

A controlled release delivery composition which can be administered to a high fat use environment such as the human gastrointestinal tract following a high fat meal. The delivery composition is embodied as a core surrounded by an asymmetric polymeric membrane. In a preferred embodiment, the asymmetric polymeric membrane is cellulose acetate.

This application is filed claiming priority to U.S. Provisional Application No. 60/432,860, filed Dec. 11, 2002.

### FIELD OF THE INVENTION

This invention relates to controlled-release of an active substance into a high fat environment such as that provided by the consumption of a high-fat meal and, more particularly, to compositions and delivery devices used therein for such controlled-release.

### BACKGROUND OF THE INVENTION

The pharmaceutical literature is replete with delivery systems for administering beneficial substances. The varied designs of such delivery systems reflect differences, for example, in desired absorption, bioavailability, and routes by which the beneficial substance (also referred to herein as a “pharmaceutical” or “active” substance or simply as a “drug”) is administered, as well as attempts to increase patient acceptability, enhance effectiveness of the active substance as delivered to its site of action, and minimization of side-effects by, for example, limiting peak blood levels.

As appreciated by those skilled in the pharmaceutical and medical arts, oral ingestion is often the preferred mode of administration given that it tends to be more convenient and less costly for the patient than other routes of administration such as, for example, intravenous, subcutaneous, and intramuscular. Moreover, the act of swallowing, versus being injected, tends to appeal much more to most patients, and is thereby more likely to ensure compliance with the dosing regimen.

Dosage forms or oral drug-delivery systems, which enable sustained-, extended-, or prolonged-release, often contain higher doses of a beneficial substance than do immediate-release preparations, and are typically designed to produce more uniform absorption of the beneficial substances delivered therefrom. Such dosage forms are referred to herein collectively as “controlled release” dosage forms.

Such controlled-release dosage forms are well known in the art. For example, beneficial substances may be incorporated into a core particle, bead, or tablet, which is coated with a polymer that controls the rate of drug release. Release mechanisms include drug diffusion through a non-porous coating, drug diffusion through a porous coating, osmotic pumping of drug controlled by the influx of water through the coating, extrusion of core contents through delivery ports in the coating by swelling of core excipients, erosion through a matrix or combinations of these mechanisms. Membrane coatings may be porous or nonporous, may contain delivery ports formed during or after the coating procedure, or may be formed in the use environment. Exemplary controlled release delivery systems are described in the following patents: U.S. Pat. No. 5,616,345, U.S. Pat. No. 5,637,320, U.S. Pat. No. 5,505,962, U.S. Pat. No. 5,354,556, U.S. Pat. No. 5,567,441, U.S. Pat. No. 5,728,402, U.S. Pat. No. 5,458,887, U.S. Pat. No. 5,736,159, U.S. Pat. No. 4,801,461, U.S. Pat. No. 5,718,700, U.S. Pat. No. 5,540,912, U.S. Pat. No. 5,612,059, U.S. Pat. No. 5,698,220, U.S. Pat. No. 4,285,987, U.S. Pat. No. 4,203,439, U.S. Pat. No. 4,116,241, U.S. Pat. No. 4,783,337, U.S. Pat. No. 4,765,989, U.S. Pat. No. 5,413,572, U.S. Pat. No. 5,324,280, U.S. Pat. No. 4,851,228, U.S. Pat. No. 4,968,507, and U.S. Pat. No. 5,366,738.

Controlled release dosage forms consisting of a drug-containing core surrounded by a rate-controlling membrane can be divided into two broad categories: diffusion delivery devices and osmotic delivery devices. For diffusion delivery devices, the active substance is released from the device by permeation from the core interior to the surrounding medium through a polymeric membrane, the primary driving force for permeation being the drug concentration difference between the interior and exterior of the dosage form. The rate of release is dependent on membrane thickness, membrane area, membrane permeability, drug concentration and solubility in the dosage form interior, and device geometry. The membrane may be dense or porous. For osmotic delivery devices, an osmotic agent (a water-swellable hydrophilic polymer or an osmogen or osmagent) is included in the device core, and the core is coated with a semipermeable membrane. The membrane may or may not include one or more delivery ports formed during membrane formation, following the coating process, or in situ. Delivery ports may range from a single large port from 0.1 to 3 mm in diameter to many small delivery ports that may consist of pores in the coating. The osmotic agent inside the core draws water into the core through the semipermeable coating. For cores containing a water-swellable hydrophilic polymer, the core imbibes water through the coating, swelling the water-swellable composition and increasing the pressure within the core, and fluidizing the drug-containing composition. Because the coating remains intact, the drug-containing composition is extruded out through the one or more delivery ports or pores in the coating into an environment of use. For cores containing an osmogen, water is osmotically drawn into the device. The increase in volume caused by the imbibition of water raises the hydrostatic pressure inside the core. This pressure is relieved by a flow of drug-containing solution or suspension out of the device through the membrane pores or a delivery port. Thus, the volume-flow rate from devices containing water-swellable polymers or osmogens is dependent on the rate of water influx through the membrane to the core. Porous, asymmetric, symmetric, or phase inversion membranes may be used to control the rate of water influx and, in turn, the rate of drug release for osmotic controlled release devices.

Such oral drug-delivery compositions necessarily reside in the fluid of the gastrointestinal tract for at least a few hours and, as a result of such prolonged presence in such fluid, may be affected by such fluid and its components unless suitably designed.

Premature disintegration, dissolution, or degradation of controlled-release oral-dosage forms in the environment of use, i.e., by the fluid of the gastrointestinal tract, and the components of such fluid, could result in uncontrolled release of the beneficial substance (either faster or slower than that desired). Hence, efforts continue toward developing materials comprising such controlled-release compositions that substantially maintain their performance despite their prolonged immersion in environments such as the fluid of the gastrointestinal tract. Ideally, drug release would be independent of variations in the composition of the GI fluid.

The prior art lists a wide variety of polymers that can be used to form coatings that control the release of the active substance from the core. See for example U.S. Pat. No. 5,616,345, U.S. Pat. No. 5,637,320, U.S. Pat. No. 5,505,962, U.S. Pat. No. 5,354,556, U.S. Pat. No. 5,567,441, U.S. Pat. No. 5,728,402, U.S. Pat. No. 5,458,887, U.S. Pat. No. 5,736,159, U.S. Pat. No. 4,801,461, U.S. Pat. No. 5,718,700, U.S. Pat. No. 5,540,912, U.S. Pat. No. 5,612,059, and U.S. Pat. No. 5,698,220. One commonly used coating material is ethyl cellulose, supplied commercially under the trade name ETHOCEL® (Dow Chemical Co.). Uses of ethyl cellulose are disclosed in, for example U.S. Pat. No. 2,853,420; Isaac Ghebre-Sellassie, Uma lyer, “Sustained-Release Pharmaceutical Micropellets Coated with Ethyl Cellulose,” Neth. Appl., 10 pp (1991); D. S. Sheorey, Sesha M. Sai, A. K. Dorle, “A New Technique for the Encapsulation of Water-Insoluble Drugs Using Ethyl Cellulose,” J. Microencapsulation, 8 (3), 359-68 (1991); A. Kristl, M. Bogataj, A. Mrhar, F. Kozjek, “Preparation and Evaluation of Ethyl Cellulose Microcapsules with Bacampicillin,” Drug Dev. Ind. Pharm., 17 (8), 1109-30 (1991); Shun Por Li, Gunvant N. Mehta, John D. Buehler, Wayne M. Grim, Richard J. Harwood, “The Effect of Film-Coating Additives on the In Vitro Dissolution Release Rate of Ethyl Cellulose-Coated Theophylline Granules,” Pharm. Technol., 14 (3), 20, 22-4 (1990); Pollock, D. K. and P. J. Sheskey, “Micronized ethylcellulose: Opportunities in Direct-Compression Controlled-Release Tablets,” Pharm. Technol. Eur. 9(1), 26-36 (1997).

It has now been determined that undesirable, uncontrolled release of beneficial substances from a controlled-release composition results, in substantial part, from the fact that compounds formed by the digestion of fatty foods present in the GI tract can act as solvents or plasticizers for the materials comprising the coatings intended for controlling the drug release from such delivery systems. In particular, such materials can swell or dissolve commonly-employed coating materials such as ethyl cellulose, thereby compromising the integrity of the coating and leading to either unacceptably slow release of drug, or unacceptably fast release of drug from the dosage form. In some cases, the contents of the use environment can lead to a substantially reduced rate of drug release, such that bioavailability is significantly, and undesirably, reduced. In other cases, the rate of drug release is substantially increased, potentially leading to dose-dumping and rapid absorption of drug by the patient, leading to undesirably high peak blood levels. Such high drug levels can potentially cause undesirable side effects or other complications.

The prior art has described dosage forms with increased, decreased, or unchanged drug delivery following a meal. Williams et al. examined the effect of peanut oil on ethyl cellulose coated dosage forms (“An In Vitro Method to Investigate Food Effects on Drug Release from Film-Coated Beads”, Williams, Sriwongjanya, and Liu, Pharmaceutical Development and Technology (1997)), and found that soaking the coated dosage forms in peanut oil prior to in vitro dissolution testing results in faster drug release for thinner coatings, and no change in drug release with thicker coatings. The same technique of soaking dosage forms in peanut oil prior to in vitro testing was used by El-Arini et al. (“Theophylline Controlled Release Preparations and Fatty Food: An In Vitro Study Using the Rotating Dialysis Cell Method”, El-Arini, Shiu, and Skelly, Pharmaceutical Research (1990)), who concluded that oil may have absorbed onto coated beads and stopped drug release by preventing wetting of the core. However, no direction was given as to how to select polymers to avoid such effects, and no indication is given of the potentially large effect of oil digestion products on coating materials.

Thus, while the prior art has described many dosage forms and coating materials for the controlled release of active substances, none have taught the use of methods for controlled-release or delivery systems which are particularly useful for controlling the release of beneficial substances while the systems are residing in a high fat environment, such as that of the fluid of the gastrointestinal tract after a high fat meal. These needs and others, which will become apparent to one skilled in the art, are met by the present invention, which is summarized and described in detail below.

### BRIEF SUMMARY OF THE INVENTION

The various aspects of the invention each, except as noted below, provide a method for the controlled-release of an active substance into a use environment, wherein said use environment comprises a substantial amount (at least about 0.5 wt %) of dietary fat.

In a first aspect, the invention provides a method for the controlled-release of an active substance into a use environment, comprising:

a. preparing a controlled-release delivery composition comprising an active-substance-containing core and an asymmetric polymeric coating thereon, wherein the polymer used to form said asymmetric polymeric coating is one which, when tested by soaking for at least 16 hours in an aqueous solution comprising 0.5 wt % dietary fat, gains less than about 15 wt %, and

b. administering said composition to said use environment,

said use environment comprising at least about 0.5 wt % of dietary fat.

“Wt%” as used above with reference to a polymer tested in a high fat environment means weight percent based on the weight of the polymer before soaking. “Wt%” as used with reference to the amount of dietary fat in a use environment means weight percent based on the weight of the components making up the environment.

“About” as used herein generally means ±20% of the number or figure it modifies.

Reference to an “asymmetric polymeric coating” is synonymous with referring to an asymmetric membrane of the type disclosed in U.S. Pat. No. 5,612,059, herein incorporated by reference. This type of membrane or coating is one which may be partially covering or all covering.

“Delivery composition” is essentially synonymous with “dosage form”. Depending on the particular release mechanism employed by the delivery composition, i.e., osmotic, diffusion, or hydrogel-driven, the delivery composition can be embodied as a bead, tablet, or capsule. If the beads are small enough, usually between 0.05 and 3 mm, they can be used as a multiparticulate for capsule fill or embodied as a powder for oral suspension, as known in the art. In general, the delivery composition is comprised of an immediate release core (or multiple cores in the case of a powder) surrounded by an asymmetric membrane through which the active substance is released in a controlled manner, by any one or more of several mechanisms, as noted above and explained and disclosed further below. Particular delivery compositions and dosage forms are described herein, and also in U.S. Pat. Nos. 5,612,059, 5,698,220, 6,068,859, and in international application PCT/IB00/01920 published as WO 01/47500, all of the preceding documents being herein incorporated by reference.

In a second aspect, the invention provides a method for the controlled-release of an active substance into a use environment, comprising:

a. preparing a controlled-release delivery composition comprising an active-substance-containing core and an asymmetric polymeric coating thereon, wherein the time to release 50% of said active substance from said composition into said use environment is at least 0.5-fold, but less than 2.0-fold the time required for said composition to release 50% of said active substance into a control use environment comprising less than about 0.1% of dietary fat, and

b. administering said composition to said use environment,

said use environment comprising at least about 0.5 wt % of dietary fat.

In a third aspect, the invention provides a method for the controlled-release of an active substance into a use environment, comprising:

a. preparing a controlled-release delivery composition comprising an active-substance-containing core and an asymmetric polymeric coating thereon, wherein the amount of drug released from said composition at any time between the 2^{nd} and 10^{th} hour following introduction of said composition to said use environment is at least 0.5-fold, but less than 2.0-fold the amount of said drug released at the same time between the 2^{nd} and 10^{th} hour by said composition into a control use environment comprising less than about 0.1% of dietary fat, and

b. administering said composition to said use environment,

said use environment comprising at least about 0.5 wt % of dietary fat.

In a fourth aspect, the invention provides a method for the controlled-release of an active substance into a use environment, comprising:

a. preparing a controlled-release delivery composition comprising an active-substance-containing core and an asymmetric polymeric coating thereon, wherein the average rate of drug release from said composition between the 2^{nd} and 10^{th} hour after introduction into said use environment is at least 0.5-fold, but less than 2.0-fold the average rate of drug release provided by said composition in a control use environment comprising less than about 0.1% of dietary fat, and

b. administering said composition to said use environment,

said use environment comprising at least about 0.5 wt % of dietary fat.

In a fifth aspect, the invention provides a method for the controlled-release of an active substance into a use environment, comprising:

a. preparing a controlled-release delivery composition comprising an active-substance-containing core and an asymmetric polymeric coating thereon, wherein the composition provides a maximum concentration of said active substance in said use environment that is at least 0.5-fold, but less than 2.0-fold the maximum concentration provided by said composition in a control use environment comprising less than about 0.1% of dietary fat, and

b. administering said composition to said use environment,

said use environment comprising at least about 0.5 wt % of dietary fat.

In a sixth aspect, the invention provides a method for the controlled-release of an active substance into a use environment, comprising:

a. preparing a controlled-release delivery composition comprising an active-substance-containing core and an asymmetric polymeric coating thereon, wherein the composition provides an area under the active substance concentration versus time curve (AUC) for any period of at least 90 minutes between the time of introduction to said use environment and about 270 minutes following introduction to said use environment that is at least 0.5-fold, but less than 2.0-fold the AUC provided by said composition in a control use environment comprising less than about 0.1% of dietary fat, and

b. administering said composition to said use environment;

said use environment comprising at least about 0.5 wt % of dietary fat.

In a seventh aspect, the invention provides a method for the controlled-release of an active substance into a use environment, comprising:

a. preparing a controlled-release delivery composition comprising an active-substance-containing core and an asymmetric polymeric coating thereon, wherein the composition provides a relative bioavailability in said use environment that is at least 0.5-fold, but less than 2.0-fold the relative bioavailability provided by said composition in a control use environment comprising less than about 0.1% of dietary fat, and

b. administering said composition to said use environment, said use environment comprising at least about 0.5 wt % of dietary fat.

In each of the seven aspects detailed above, a preferred embodiment of the invention occurs when the use environment contains at least 2 wt % of dietary fat.

A controlled release delivery composition which exhibits one or more of the above-noted seven aspects (i.e. as set forth in section (a) of each aspect) is considered within the scope of the invention.

In an eighth aspect, the invention provides a therapeutic package, comprising: a container, a controlled-release delivery composition for the controlled release of an active substance as disclosed and described in section (a) of any of the previous seven aspects described above, and, associated with said package, written matter non-limited as to whether the dosage form can be taken with or without food, particularly high fat food. In this aspect, written matter associated with the package used to store, transport, and/or vend the controlled release delivery compositions of this invention, whether the written matter is of a regulatory, non-regulatory informational (e.g., advertising) or other language associated with the package can not, within the scope of the invention, direct that the dosage forms therein are not to be taken with food. Thus the package as described above excludes, for example, therapeutic packages containing a package insert containing a regulatory-required warning such as “do not administer more than one hour before a meal up to two hours after a meal”, or similar language imparting the same warning.

As used herein, the term “a controlled-release delivery composition” is essentially synonymous with “a controlled-release dosage form”.

Reference above to a “control” or to a “control use environment” means an environment which, whether in vivo or in vitro, is, or which substantially mimics, the GI tract when it does not contain a substantial amount of dietary fat. By “does not contain a substantial amount of dietary fat” is meant that the control use environment is essentially free from dietary fat. In general, this means that the control environment contains less than 0.1 wt % of dietary fat.

With respect to the range “0.5 to 2.0-fold” wherever expressed above (i.e. in each of the (a) sections of the first seven aspects), a preferred subrange is 0.75-fold to 1.5-fold. A more preferred range is 0.8-fold to 1.25-fold.

Terms such as “drug”, “therapeutic agent”, “active substance”, “active pharmaceutical agent”, and “beneficial agent” are used interchangeably herein.

The various aspects of the present invention each provide one or more of the following advantages. The methods of the present invention provide reliable, safe controlled-release of an active substance to a use environment that is independent of the fed/fasted state of a patient or the nature of the food ingested by the patient in need of therapy of the active substance. The present invention also minimizes the potential for dose dumping or incomplete drug delivery due to dissolution or plasticization of the polymeric coating, minimizing the possibility of high-blood levels and resulting adverse effects.

The controlled release dosage forms disclosed herein comprise, as described above, drug-containing core which is surrounded by an asymmetric polymeric rate-limiting membrane that imparts the desired controlled release characteristics to the overall dosage form. That is, in the absence of the polymeric rate-limiting coating, the core would effect more rapid release of active substance than when coated with an asymmetric coating. The dosage form can comprise additional components as known in the art, which components contribute to embodiments that form part of this invention. For example, the dosage form may further comprise a film coating or taste-masking coating surrounding the rate-limiting membrane. Alternatively, In some cases, a coating of immediate release drug may be formed surrounding the rate-limiting membrane to supply an immediate bolus of drug in addition to drug which is released in a controlled release manner.

### DETAILED DESCRIPTION OF THE INVENTION

The present invention provides a method for the controlled-release of an active substance into a use environment, wherein said use environment comprises a substantial amount of dietary fat during a substantial part of said release and wherein the active substance is delivered via a controlled-release composition. As described above in the Background, the inventors have found that fatty foods, and in particular, the digestion products of dietary fat present in the use environment can act as solvents or plasticizers for the materials comprising the rate-controlling coatings of such controlled-release compositions. Accordingly, the method of the present invention comprises preparing controlled-release compositions and then administering such compositions to a use environment containing a substantial amount (at least about 0.5 wt %) of dietary fat such that the rate of release of active substance from the composition is about the same as that of the composition in a control use environment that does not contain a substantial amount of dietary fat (i.e., 0.1 wt % or less).

Use Environment

Reference to the “release” of drug as used herein means transport of drug from the interior of the delivery composition to its exterior such that it contacts the fluid of a use environment. Reference to a “use environment” can either be in vivo GI fluids or an in vitro test medium. “Administration” to a use environment includes either by ingestion or swallowing, where the use environment is in vivo, or being placed in a test medium where the use environment is in vitro.

Drug release, given in wt %, refers to the mass of drug released divided by the total mass of drug initially in the composition multiplied by 100. As used here and in the claims, the average rate of drug release per hour for a time period is defined as the wt % drug released during the time period divided by the duration (in hours) of the time period.

The term “fat” is used herein as having its conventional, art-recognized meaning of the biological substance comprised primarily of triglycerides, but which may also comprise minor portions of di- and mono-glycerides as well.

In the method of the present invention, the active substance is released to a use environment containing a substantial amount of dietary fat during a substantial part of the time that the controlled-release delivery composition is present in the use environment. “Dietary fat” as used herein can have an in vivo or in vitro meaning, depending on context; that is, depending on whether the reference to “dietary fat” is a reference to dietary fat in the gastrointestinal tract (in vivo) or to artificial dietary fat created for purposes of making an artificial high fat environment (in vitro) or a low fat control environment (in vitro) which, for purposes of this invention, mimics the characteristics and release behavior of the human GI tract. Thus, “dietary fat” can mean fats, including fat digestion products, i.e., the products of fat metabolism by enzymes in the human GI tract. “Dietary fat” also embraces the fat and fat hydrolysis products artificially produced (i.e., to mimic in vivo fat and fat digestion products) for use in the in vitro tests disclosed herein for use in helping to define the invention.

In in vivo tests, the use environment generally refers to the gastrointestinal tract of an animal, including that of a human. An in vivo use environment containing a substantial amount of dietary fat is generated by having the subject ingest a meal containing dietary fat less than about 4 hours prior to, during, or less than about 2 hours after administration of the delivery composition to the subjects' gastrointestinal tract. An appropriate meal containing dietary fat is a standard “FDA high-fat breakfast.” A standard “FDA high-fat breakfast” consists of 2 eggs fried in butter, 2 strips of bacon, 2 slices of toast with butter, 4 ounces of hash brown potatoes, 8 ounces of whole milk (i.e., approximately 150 protein calories, 250 carbohydrate calories, 500-600 fat calories). Alternative meals with equivalent nutritional content can be used. The high-fat meal contains about 50 to 60 gm of fat. Thus, once ingested, the concentration of fat in the use environment ranges anywhere from about 0.5 wt % or higher based on the total weight of the breakfast or meal and the weight of fluid in the GI tract. Thus, “a substantial amount” of dietary fat means that the use environment contains greater than about 0.5 wt % dietary fats, based on the total weight of the breakfast or meal.

When referring herein to in vivo measurements in, as the use environment, the GI tract, such measurements are made, inter alia, by analyzing the concentration of active substance per unit volume of the plasma or blood. The concentration of active substance in blood or plasma is assumed to be proportional to the concentration in the GI tract. The actual in vivo data collected is at least one, and usually several or even numerous data points, each reflecting the concentration of active substance thus measured in blood or plasma corresponding to the particular time interval which elapses between the time the dosage form is swallowed and the time the blood or plasma is withdrawn from the patient. Such data points may be used individually (see, for example, claim 3 in which only a single measurement is required). Alternatively, such data points may be used to construct an AUC, as conventionally known in the art (see, for example, claim 6) or to calculate an average (see, for example, claim 4). Thus, a composition of the present invention can be determined by measuring the amount of active substance released into the use environment, or by measuring the concentration of active substance in the plasma or blood.

In in vitro tests, it is preferred that the use environment mimic the partially-digested dietary fats (fats and fat hydrolysis products) present in in vivo tests. One such in vitro use environment is a “standard blended breakfast mixed with simulated intestinal fluid containing enzymes” (“SBB/SIF”) test liquid. The SBB/SIF solution is prepared as follows. First, 6.8 g of monobasic potassium phosphate is dissolved in 250 mL of water. Next, 190 mL of 0.2 N sodium hydroxide is mixed with 400 mL of water and combined with the potassium phosphate solution. Next, 10 g of pancreatin is added, and the pH of the resulting solution adjusted to 7.5±0.1 with 0.2 N sodium hydroxide. Water is then added for a final volume of 1000 mL. To 250 mL of this solution is then added the standard “FDA high-fat breakfast” defined above. The solution is then blended at high speed to reduce the particle size to form the SBB/SIF test liquid. The SBB/SIF solution is then kept at 37° C. for at least 10 minutes and no more than 60 minutes before use in in vitro tests. The resulting SBB/SIF solution contains at least about 0.5 wt % dietary fats based on the weight of the solution.

Alternatively, an in vitro use environment containing a substantial amount of dietary fat (i.e. at least about 0.5 wt %) can be formed by forming an aqueous suspension or emulsion containing a mixture of oils and other compounds designed to mimic partially-digested dietary fats. One such mixture of oils is a “50% hydrolyzed model oil.” By “50% hydrolyzed model oil” is meant an oil mixture containing 38 wt % olive oil (Sigma Diagnostics, St. Louis, Mo.), 15 wt % glyceryl monooleate (Myverol® 18-99, Eastman Chemical Co., Kingsport, Tenn.), 23 wt % oleic acid (Aldrich Chemical Co., Milwaukee, Wis.), 9 wt % tripalmitin (Sigma, St. Louis, Mo.), 4 wt % glyceryl monostearate (Imwitor® 191, HULS America Inc., Piscataway, N.J.), 5 wt % palmitic acid (Sigma), 3 wt % tributyrin (Sigma), 2 wt % butyric acid (Aldrich Chemical Co.), and 1 wt % lecithin (Sigma). The 50% hydrolyzed model oil can be added to an appropriate aqueous solution to form a use environment containing a substantial amount of dietary fat. One suitable aqueous solution is a simulated gastric buffer comprising 0.01 M HCl. Another suitable aqueous solution is a Phosphate Buffered Saline (“PBS”) solution, comprising 20 mM sodium phosphate (Na₂HPO₄), 47 mM potassium phosphate (KH₂PO₄), 87 mM NaCl, and 0.2 mM KCl, adjusted to pH 6.5 with NaOH. Another suitable aqueous solution is a Model Fasted Duodenal (“MFD”) solution, comprising the above PBS solution to which has been added 7.3 mM sodium taurocholic acid and 1.4 mM 1-palmitoyl-2-oleyl-sn-glycero-3-phosphocholine, adjusted to pH 6.5.

The 50% hydrolyzed model oil should be added to an appropriate aqueous solution at a concentration that mimics the concentration of dietary fat in in vivo tests. Thus, one suitable in vitro use environment consists of 0.5 wt % 50% hydrolyzed model oil in simulated gastric buffer comprising 0.01 M HCl.

The following in vitro tests are described as being predictive of polymer behavior that would be observed in humans having just imbibed a high fat breakfast comprising at least 0.5 wt % dietary fat.

An in vitro test may be used to evaluate a dosage form of the invention. In a preferred method, dosage forms to be tested are added to a round bottom flask containing 100 mL of a receptor solution (i.e., of a simulated use environment, such as MFD, SBB/SIF, or aqueous solution containing 50% hydrolyzed model oil). Suitable receptor solutions are the use environments described above for in vitro tests. The round bottom flask is affixed with a holder attached to a rotating wheel, which is maintained at 37° C. Samples are rotated at 37° C., preferably for 6 hours, then analyzed by visual examination of the core. Residual analysis is performed to determine the amount of drug remaining in the core, and drug release is calculated by difference.

An alternative in vitro test is a direct test, in which samples of the dosage form are placed into a stirred USP Type II dissolution flask containing the receptor solution. Tablets are placed in a wire support, paddle height is adjusted, and the dissoette flasks stirred at 50 rpm at 37° C. Samples are taken at periodic intervals using a VanKel VK8000 autosampling dissoette with automatic receptor solution replacement. The autosampler dissoette device is programmed to periodically remove a sample of the receptor solution, and the drug concentration is analyzed by HPLC.

It is noted that if it is intended to effect a comparison of release characteristics as between different dosage forms, the same in vitro fat-containing dissolution test media should be used. Stated differently, if a test of a first dosage form or composition is conducted in SBB/SIF solution, then the testing of a second and any other comparison test dosage forms should be conducted in the same or identical in vitro fat-containing test solution. When conducting the control portion of such a comparison, i.e., of different dosage forms in a control use environment (i.e., containing no fat), any of the (non-fat containing) test media will work for purposes of the present invention. For assessing control dissolution profiles, it is preferred, for the sake of consistency, simply to use the same dissolution medium as that used as the fat-containing dissolution test medium, except that the control medium contains no fat.

Alternatively, an in vivo test may be used to evaluate a dosage form of the invention. However, due to the relative complexity and cost of the in vivo procedure, it is preferred that in vitro procedures be used to evaluate dosage forms even though the ultimate use environment is usually the human GI tract. In in vivo tests, drug dosage forms are dosed to a group of animals, such as humans or dogs, and drug release and drug absorption is monitored either by (1) periodically withdrawing blood and measuring the serum or plasma concentration of drug or periodically measuring the drug concentration in the urine or (2) measuring the amount of drug remaining in the dosage form following its exit from the anus (residual drug) or (3) both (1) and (2). In the second method, residual drug is measured by recovering the tablet upon exit from the anus of the test subject and measuring the amount of drug remaining in the dosage form using the same procedure described above for the in vitro residual test. The difference between the amount of drug in the original dosage form and the amount of residual drug is a measure of the amount of drug released during the mouth-to-anus transit time. The control is preferably crossed over, i.e., it is the same group of animals dosed after having fasted for at least 8 hours, and which continues fasting for at least 4 hours after dosing. This test has limited utility since it provides only a single drug release time point but is useful in demonstrating the correlation between in vitro and in vivo release. The aforementioned data is used to measure active substance released into an in vivo use environment.

In one in vivo method of monitoring drug release and absorption, the serum or plasma drug concentration is plotted along the ordinate (y-axis) against the blood sample time along the abscissa (x-axis). The data may then be analyzed to determine drug release rates using any conventional analysis, such as the Wagner-Nelson or Loo-Riegelman analysis. See also Welling, “Pharmacokinetics: Processes and Mathematics” (ACS Monograph 185, Amer. Chem. Soc., Washington, D.C., 1986). Treatment of the data in this manner yields an apparent in vivo drug release profile.

In any of the in vivo or in vitro tests disclosed above, a dosage form which passes (i.e., produces at least the result called for in the claims, within experimental error) any one or more of the tests is considered to be within the scope of the claims.

The Drug

The drug may be virtually any beneficial therapeutic agent and may comprise from 0.1 to 90 wt % of the core. The drug may be in any form, either crystalline or amorphous. The drug may also be in the form of a solid dispersion. The drug may be employed in its neutral (e.g., free acid or free base) form, or in the form of its pharmaceutically acceptable salts as well as in anhydrous, hydrated, or solvated forms, and pro drugs.

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, and cholesterol ester transfer protein inhibitors.

Each named drug should be understood to include the neutral or ionized form of the drug, pharmaceutically acceptable salts, as well as prodrugs. Specific examples of antihypertensives include prazosin, nifedipine, amlodipine besylate, trimazosin and doxazosin; specific examples of blood glucose-lowering agents are glipizide and chlorpropamide; specific example of anti-impotence agents are sildenafil and sildenafil citrate; specific examples of antineoplastics include chlorambucil, lomustine and echinomycin; a specific example of an imidazole-type antineoplastic is tubulazole; a specific example of an antihypercholesterolemic is atorvastatin calcium; specific examples of anxiolytics include hydroxyzine hydrochloride and doxepin hydrochloride; specific examples of anti-inflammatory agents include betamethasone, prednisolone, aspirin, piroxicam, valdecoxib, carprofen, celecoxib, flurbiprofen and (+)-N-{4-[3-(4-fluorophenoxy)phenoxy]-2-cyclopenten-1-yl}-N-hyroxyurea; a specific example of a barbiturate is phenobarbital; specific examples of antivirals include acyclovir, nelfinavir, and virazole; specific examples of vitamins/nutritional agents include retinol and vitamin E; specific examples of beta blockers include timolol and nadolol; a specific example of an emetic is apomorphine; specific examples of a diuretic include chlorthalidone and spironolactone; a specific example of an anticoagulant is dicumarol; specific examples of cardiotonics include digoxin and digitoxin; specific examples of androgens include 17-methyltestosterone and testosterone; a specific example of a mineral corticoid is desoxycorticosterone; a specific example of a steroidal hypnotic/anesthetic is alfaxalone; specific examples of anabolic agents include fluoxymesterone and methanstenolone; specific examples of antidepression agents include sulpiride, [3,6-dimethyl-2-(2,4,6-trimethyl-phenoxy)-pyridin-4-yl]-(1-ethylpropyl)-amine, 3,5-dimethyl-4-(3′-pentoxy)-2-(2′,4′,6′-trimethylphenoxy)pyridine, pyroxidine, fluoxetine, paroxetine, venlafaxine and sertraline; specific examples of antibiotics include carbenicillin indanylsodium, bacampicillin hydrochloride, troleandomycin, doxycyline hyclate, ampicillin and penicillin G; specific examples of anti-infectives include benzalkonium chloride and chlorhexidine; specific examples of coronary vasodilators include nitroglycerin and mioflazine; a specific example of a hypnotic is etomidate; specific examples of carbonic anhydrase inhibitors include acetazolamide and chlorzolamide; specific examples of antifungals include econazole, terconazole, fluconazole, voriconazole, and griseofulvin; a specific example of an antiprotozoal is metronidazole; specific examples of anthelmintic agents include thiabendazole, oxfendazole and morantel; specific examples of antihistamines include astemizole, levocabastine, cetirizine, loratadine, decarboethoxyloratadine and cinnarizine; specific examples of antipsychotics include ziprasidone, olanzepine, thiothixene hydrochloride, fluspirilene, risperidone and penfluridole; specific examples of gastrointestinal agents include loperamide and cisapride; specific examples of serotonin antagonists include ketanserin and mianserin; a specific example of an anesthetic is lidocaine; a specific example of a hypoglycemic agent is acetohexamide; a specific example of an anti-emetic is dimenhydrinate; a specific example of an antibacterial is cotrimoxazole; a specific example of a dopaminergic agent is L-DOPA; specific examples of anti-Alzheimer's Disease agents are THA and donepezil; a specific example of an anti-ulcer agent/H2 antagonist is famotidine; specific examples of sedative/hypnotic agents include chlordiazepoxide and triazolam; a specific example of a vasodilator is alprostadil; a specific example of a platelet inhibitor is prostacyclin; specific examples of ACE inhibitor/antihypertensive agents include enalaprilic acid and lisinopril; specific examples of tetracycline antibiotics include oxytetracycline and minocycline; specific examples of macrolide antibiotics include erythromycin, clarithromycin, and spiramycin; a specific example of an azalide antibiotic is azithromycin, specific examples of glycogen phosphorylase inhibitors include [R—(R*S*)]-5-chloro-N-[2-hydroxy-3-{methoxymethylamino}-3-oxo-1-(phenylmethyl)propyl-1H-indole-2-carboxamide and 5-chloro-1H-indole-2-carboxylic acid [(1S)-benzyl-(2R)-hydroxy-3-((3R,4S)-dihydroxypyrrolidin-1-yl-)-3-oxypropyl]amide; specific examples of cholesterol ester transfer protein inhibitors include [2R,4S]-4-[3,5-bis-trifluoromethyl-benzyl)-methoxycarbonyl-amino]-2-ethyl-6-trifluoromethyl-3,4-dihydro-2H-quinoline-1-carboxylic acid ethyl ester and [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester.

The drug may be present in the form of a solid, amorphous dispersion. By solid, amorphous dispersion is meant that the drug is dispersed in a polymer so that a major portion of the drug is in a substantially amorphous or non-crystalline state, and its non-crystalline nature is demonstrable by x-ray diffraction analysis or by differential scanning calorimetry. The dispersion may contain from about 5 to 90 wt % drug, preferably 10 to 70 wt %. The polymer is soluble in aqueous media and inert. Suitable polymers and methods for making solid amorphous dispersions are disclosed in commonly assigned patent application Ser. No. 09/495,061 filed Jan. 31, 2000 (which claims priority date of the provisional patent application Ser. No. 60/119,406 filed Feb. 10, 1999), the relevant disclosure of which is incorporated by reference. Suitable dispersion polymers include ionizable and non-ionizable cellulosic polymers, such as cellulose esters, cellulose ethers, and cellulose esters/ethers; and vinyl polymers and copolymers having substituents selected from the group consisting of hydroxyl, alkylacyloxy, and cyclicamido, such as polyvinyl pyrrolidone, polyvinyl alcohol, copolymers of polyvinyl pyrrolidone and polyvinyl acetate. Particularly preferred polymers include hydroxypropylmethyl cellulose acetate succinate (HPMCAS), hydroxypropyl methyl cellulose (HPMC), hydroxypropyl methyl cellulose phthalate (HPMCP), cellulose acetate phthalate (CAP), cellulose acetate trimellitate (CAT), and polyvinyl pyrrolidone (PVP). Most preferred are HPMCAS, HPMCP, CAP and CAT.

The Core

The controlled-release delivery compositions used in the present invention comprise a drug incorporated into an immediate release core particle, bead, or tablet, which is coated with an asymmetric rate-limiting coating. The dosage form can be engineered so that the release mechanism involves drug diffusion through the asymmetric coating, osmotic pumping of drug controlled by the influx of water through the asymmetric coating, extrusion of core contents through delivery ports in the coating by swelling of core excipients, osmotic bursting of the coating due to influx of water into the core or combinations of these mechanisms. As previously disclosed and further explained below, any coating used in the invention is asymmetric. Asymmetric membrane coatings may be porous or nonporous, or may contain delivery ports formed during or after the coating procedure, or may be formed in the use environment. Details of the drug, core, and coating are discussed below.

The core generally comprises the drug and other excipients required for the type of delivery mechanism desired. The invention is suitable for use with osmotic devices, hydrogel-driven devices, and diffusion devices, described in detail below.

Osmotic Devices

In one embodiment, the controlled-release dosage form has two components: (a) a core containing the drug; and (b) a non-dissolving and non-eroding asymmetric coating surrounding the core, the asymmetric coating controlling the influx of water to the core from an aqueous environment of use so as to cause drug release by extrusion of some or all of the core to the environment of use. Osmotic drug-delivery devices are described in the following U.S. Pat. Nos. 5,612,059, 5,698,220, 5,728,402, 5,458,887, 5,736,159, 5,654,005, 5,558,879, 4,801,461, 4,285,987, 4,203,439, 4,116,241, international application PCT/IB00/01920 published as WO 01/47500, and patent application Ser. No. 09/495,061 filed Jan. 31, 2000 (which claims priority of provisional patent application Ser. No. 60/119,406 filed Feb. 10, 1999), the pertinent disclosures of which are incorporated herein by reference.

The term “extrusion” as it relates to the drug delivery mechanism is intended to convey an expulsion or forcing out of some or all of the core through at least one delivery port. By “at least one delivery port” is meant one or more holes, slits, passageways, channels or pores that may range in size from 0.1 to more than 3000 μm in diameter that permit release of drug from the dosage form. The drug may be delivered by the extrusion either in the form of a suspension of solids in water or primarily as a solution of the drug, to the extent dissolution has taken place in the core.

In addition to the drug, the core includes an “osmotic agent.” By “osmotic agent” is meant any agent that creates a driving force for transport of water from the environment of use into the core of the device. Exemplary osmotic agents are water-swellable hydrophilic polymers and osmotically-effective solutes. Thus, the core may include water-swellable hydrophilic polymers, both ionic and nonionic, often referred to as “osmopolymers” and “hydrogels.” The amount of water-swellable hydrophilic polymers present in the core may range from about 5 to about 80 wt %, preferably 10 to 50 wt %. Exemplary materials include hydrophilic vinyl and acrylic polymers, polysaccharides such as calcium alginate, PEO, PEG, PPG, poly(2-hydroxyethyl methacrylate), poly(acrylic) acid, poly(methacrylic) acid, PVP and crosslinked PVP, PVA, PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, HEC, HPC, HPMC, CMC and CEC, sodium alginate, polycarbophil, gelatin, xanthan gum, and sodium starch glycolate. Other materials include hydrogels comprising interpenetrating networks of polymers which may be formed by addition or by condensation polymerization, the components of which may comprise hydrophilic and hydrophobic monomers such as those just mentioned. Preferred polymers for use as the water-swellable hydrophilic polymers include PEO, PEG, PVP, sodium croscarmellose, HPMC, sodium starch glycolate, polyacrylic acid and crosslinked versions or mixtures thereof.

By “osmotically effective solutes,” is meant any water-soluble compound that is commonly referred to in the pharmaceutical arts as an “osmogen” or an “osmagent.” The amount of osmogen present in the core may range from about 2 to about 70 wt %, preferably 10 to 50 wt %. Typical classes of suitable osmogens are water-soluble organic acids, salts and sugars that are capable of imbibing water to thereby effect an osmotic pressure gradient across the barrier of the surrounding coating. Typical useful osmogens include magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. Particularly preferred osmogens are glucose, lactose, sucrose, mannitol, xylitol and sodium chloride. When the drug has sufficient aqueous solubility, the drug itself may act as an osmogen.

Finally, the core may include a wide variety of additives and excipients that enhance drug solubility or that promote stability, tableting or processing of the dispersion. Such additives and excipients include tableting aids, surfactants, water-soluble polymers, pH modifiers, fillers, binders, pigments, disintegrants, antioxidants, lubricants and flavorants. Examples of such components are microcrystalline cellulose; metallic salts of acids such as aluminum stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmiticstearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxethylene glycols, and polytetrafluoroethylene; and inorganic materials such as talc, dicalcium phosphate, and silicon dioxide; sugars such as lactose and xylitol; and sodium starch glycolate.

The core may also include solubility-enhancing agents that promote the water solubility of the drug, present in an amount ranging from about 5 to about 50 wt %. Examples of suitable solubility-enhancing agents include surfactants; pH control agents such as buffers, organic acids and organic acid salts and organic and inorganic bases; glycerides; partial glycerides; glyceride derivatives; polyoxyethylene and polyoxypropylene ethers and their copolymers; sorbitan esters; polyoxyethylene sorbitan esters; carbonate salts; alkyl sulfonates; and cyclodextrins.

In a particular osmotic embodiment, a “homogeneous core device”, the core can consist of one or more pharmaceutically active agents, water-soluble compounds for inducing osmosis, non-swelling solubilizing agents, non-swelling (water-soluble or water-insoluble) wicking agents, swellable hydrophilic polymers, binders and lubricants. Such devices are disclosed in U.S. Pat. Nos. 5,516,527 and 5,792,471, herein incorporated by reference.

The osmotically active (water-soluble) agent is typically a sugar alcohol such as mannitol or sorbitol, or sugars in combination with polysaccharides such as dextrose and maltose, or a physiologically tolerable ionic salt which is compatible with the other components such as sodium or potassium chloride, or urea. Examples of water-soluble compounds for inducing osmosis are: inorganic salts such as magnesium chloride or magnesium sulfate, lithium, sodium or potassium chloride, lithium, sodium or potassium hydrogen or dihydrogen phosphate, salts of organic acids such as sodium or potassium acetate, magnesium succinate, sodium benzoate, sodium citrate or sodium ascorbate; carbohydrates such as sorbitol or mannitol (hexite), arabinose, dextrose, ribose or xylose (pentosene), glucose, fructose, galactose or mannose (hexosene), sucrose, maltose or lactose (disacharides) or raffinose (trisacharides); water-soluble amino acids such as glycine, leucine, alanine or methionine, urea and the like, and mixtures thereof. These water-soluble excipients may be present in the core in amounts by weight of about 0.01 to 45%, based on the total weight of the dosage form.

Non-swelling solubilizing agents include (a) agents that inhibit crystal formation of the pharmaceutical or otherwise act by complexation therewith; (b) high HLB (hydrophilic-lipophilic balance) micelle-forming surfactants, particularly non-ionic and/or anionic surfactants: (c) citrate esters; and combinations thereof, particularly combinations of complexing agents and anionic surfactants. Examples of agents that inhibit crystal formation of the pharmaceutical or otherwise acts by complexation therewith include polyvinylpyrrolidone, polyethyleneglycol (particularly PEG 8000), cyclodextrins and modified cyclodextrins. Examples of high HLB, micelle forming surfactants include Tween 20, Tween 60, Tween 80, polyoxyethylene or polyethylene-containing surfactants, or other long chain anionic surfactants, particularly sodium lauryl sulfate. Examples of citrate ester derivatives that are preferred are the alkyl esters, particularly triethyl citrate. Combinations of these which are particularly preferred are polyvinylpyrrolidone with sodium lauryl sulfate and polyethyleneglycol with sodium lauryl sulfate.

Non-swelling wicking (wetting) agents are used to create channels or pores in the core of the tablet. This facilitates channeling of water through the core by physisorption. Preferred wicking agents do not swell to any appreciable degree. These materials can be water soluble or water insoluble materials. Water-soluble materials suitable for acting as wicking (wetting) agents include surface-active compounds, i.e., surfactants, e.g. anionic surfactants of the alkylsulfate type such a sodium, potassium or magnesium lauryl sulfate, n-tetradecylsulfate, n-hexadecyl sulfate or n-octadecylsulfate; or of the alkyl ether sulfate type, e.g., sodium, potassium or magnesium n-dodecyloxyethyl sulfate, n-tetradecyloxyethyl sulfate, n-hexadecyloxyethyl sulfate or n-octadecyloxyethyl sulfate; or of the alkylsulfonate type, e.g., sodium potassium or magnesium n-dodecanesulfonate, n-tetradecanesulfonate, n-hexadecanesulfonate or n-octadecanesulfonate. Further suitable surfactants are nonionic surfactants of the fatty acid polyhydroxy alcohol ester type such as sorbitan monolaurate, sorbitan tristerate or trioliate, polyethylene glycol fatty acid ester such as polyoxyethyl stearate, polyethylene glycol 400 stearate, polyethylene glycol 2000 stearate, preferably ethylene oxide/propylene oxide block copolymers of the Pluronics (BWC) or Synperionic (ICI) type, polyglycerol-fatty acid esters or glyceryl-fatty acid esters. Especially suitable is sodium lauryl sulfate. When present, these surfactants should be preferably present from about 0.2 to 2% based on the total core weight. Other soluble wicking (wetting) agents include low molecular weight polyvinyl pyrrolidone and n-pyrol.

Insoluble materials suitable for acting as wicking (wetting) agents include, but are not limited to, colloidal silicon dioxide, kaolin, titanium dioxide, fumed silicon dioxide, alumina, niacinamide, bentonite, magnesium aluminum silicate, polyester, polyethylene. Particularly suitable insoluble wicking agents include colloidal silicon dioxide.

In a further particular osmotic embodiment, a “bursting osmotic core device”, the active therapeutic agent is incorporated into a tablet core or bead core containing the agent and one or more osmagents. Devices of this type have been generally disclosed in Baker, U.S. Pat. No. 3,952,741, which is incorporated herein by reference. Examples of osmagents are sugars such as glucose, sucrose, mannitol, lactose, and the like; and salts such as sodium chloride, potassium chloride, sodium carbonate, and the like; water-soluble acids such as tartaric acid, fumaric acid, and the like. The device core is coated with a polymer which forms a semipermeable membrane, that is, a membrane which is permeable to water but is substantially impermeable to the therapeutic agent. An example of a preferred polymer which provides a semipermeable membrane is cellulose acetate.

When a coated tablet or bead of the “bursting osmotic core” embodiment described above is placed in an aqueous environment of use, water passes through the semipermeable membrane into the core, dissolving a portion of the therapeutic agent and osmagent, generating a hydrostatic pressure which results in bursting of the semipermeable membrane and release of therapeutic agent into the aqueous environment. By choice of bead or tablet core size and geometry, identity and quantity of osmagent, and thickness of the semipermeable membrane, the time lag between placement of the dosage form into the aqueous environment of use and release of the enclosed agent may be chosen. It will be appreciated by those skilled in the art that increasing the surface-to-volume ratio of the dosage form, and increasing the osmotic activity of the osmagent serve to decrease the time lag, whereas increasing the thickness of the coating will increase the time lag. A bursting osmotic core tablet or bead has a tablet or bead core which may contain from about 25-95% therapeutic agent, about 0-60% osmagent, as described above, and about 5-20% other pharmaceutical aids such as fillers, binders and lubricants. The semipermeable membrane coating on a tablet, preferably a cellulose acetate coating, is present at a weight corresponding to from about 2% to about 30%, preferably from about 3% to about 10%, of the weight of the tablet core. The semipermeable membrane coating on a bead, preferably a cellulose acetate coating, is present at a weight corresponding to from about 2% to about 80%, preferably from 3% to 30%, of the weight of the bead core.

In a further embodiment, a “bursting coated swelling core”, a therapeutic agent-containing tablet or bead is prepared which, in addition to osmagents, also comprises 15-70% of a swellable material, such as a swellable colloid (e.g., gelatin), as described in Milosovich, U.S. Pat. No. 3,247,066, incorporated herein by reference. Preferred swelling core materials are hydrogels, i.e., hydrophilic polymers which take up water and swell, such as polyethylene oxides, polyacrylic acid derivatives such as polymethyl methacrylate, polyacrylamides, polyvinyl alcohol, poly-N-vinyl-2-pyrrolidone, carboxymethylcellulose, starches, and the like. Preferred swelling hydrogels for this embodiment are polyethylene oxides and carboxymethylcellulose. The colloid/hydrogel-containing, therapeutic agent-containing core tablet or bead is coated, at least in part, by a semipermeable membrane.

When a coated tablet or bead having a bursting coated swelling core is placed in an aqueous environment of use, water passes through the semipermeable membrane into the core, swelling the core and resulting in bursting of the semipermeable membrane and release of the therapeutic agent into the aqueous environment.

Hydrogel-Driven Devices

In another embodiment, the drug-containing core comprises two compositions: a drug-containing composition and a water-swellable composition. Hydrogel driven devices operate similarly to osmotic devices, the main difference being that the drug-containing composition and the water-swellable composition in a hydrogel-driven device occupy separate regions in the core. By “separate regions” is meant that the two compositions occupy separate volumes, such that the two are not substantially mixed together. An asymmetric coating surrounds the core and is water-permeable, water-insoluble and has one or more delivery ports therethrough. In use, the core imbibes water through the coating from the environment of use such as the gastrointestinal (“GI”) tract. The imbibed water causes the water-swellable composition to swell, thereby increasing the pressure within the core. The imbibed water also increases the fluidity of the drug-containing composition. The pressure difference between the core and the environment of use drives the release of the fluidized drug-containing composition. Because the coating remains intact, the drug-containing composition is extruded out of the core through the delivery port(s) into the environment of use. Because the water-swellable composition contains no drug, almost all of the drug is extruded through the delivery port(s), leaving very little residual drug. Such hydrogel-driven devices are disclosed in U.S. Pat. Nos. 5,718,700, 4,783,337, 4,765,989, 4,865,598, 5,273,752, and U.S. application Ser. No. 09/745,095, filed Dec. 20, 2000, the full disclosures of which are incorporated herein by reference.

In addition to the drug, the drug-containing composition may comprise osmotic agents, tableting aids, surfactants, water-soluble polymers, pH modifiers, fillers, binders, pigments, disintegrants, antioxidants, lubricants, flavorants, and solubility-enhancing agents as described above for Osmotic Devices. In addition, the drug-containing composition may further comprise entraining agents and/or fluidizing agents. Entraining agents are especially preferred for delivery of low solubility drugs. They suspend or entrain the drug so as to aid in the delivery of the drug through the delivery port(s) to the environment of use. The amount of the entraining agent present in the drug-containing composition may range from about 20 wt % to about 98 wt % of the drug-containing composition. The entraining agent may be a single material or a mixture of materials. Examples of such materials include polyols, and oligomers of polyethers, such as ethylene glycol oligomers or propylene glycol oligomers. In addition, mixtures of polyfunctional organic acids and cationic materials such as amino acids or multivalent salts, such as calcium salts may be used. Of particular utility are polymers such as polyethylene oxide (PEO), polyvinyl alcohol, PVP, cellulosics such as hydroxyethyl cellulose (HEC), hydroxypropylcellulose (HPC), HPMC, methyl cellulose (MC), carboxy methyl cellulose (CMC), carboxyethylcellulose (CEC), gelatin, xanthan gum or any other water-soluble polymer that forms an aqueous solution with a viscosity similar to that of the polymers listed above. An especially preferred entraining agent is non-crosslinked PEO or mixtures of PEO with the other materials listed above.

The drug-containing composition may further comprise a fluidizing agent. As used herein, a “fluidizing agent” is a water-soluble compound that allows the drug-containing composition to rapidly become fluid upon imbibing water when the dosage form is introduced into a use environment. The fluidizing agent can be essentially any water-soluble compound that rapidly increases the fluidity of the drug-containing composition when water is imbibed into the core. Exemplary fluidizing agents are sugars, organic acids, amino acids, polyols, salts, and low-molecular weight oligomers of water-soluble polymers. Exemplary sugars are glucose, sucrose, xylitol, fructose, lactose, mannitol, sorbitol, maltitol, and the like. Exemplary organic acids are citric acid, lactic acid, ascorbic acid, tartaric acid, malic acid, fumaric, and succinic acid. Exemplary amino acids are alanine and glycine. Exemplary polyols are propylene glycol and sorbitol. Exemplary oligomers of low-molecular weight polymers are polyethylene glycols with molecular weights of 10,000 daltons or less. Particularly preferred fluidizing agents are sugars and organic acids. Such fluidizing agents are preferred as they often improve tableting and compression properties of the drug-containing composition relative to other fluidizing agents such as inorganic salts or low-molecular weight polymers.

The core further comprises a water-swellable composition. The water-swellable composition greatly expands as it imbibes water through the coating from the use environment. As it expands, the water-swellable composition increases the pressure within the core, causing extrusion of the fluidized drug-containing composition through the port(s) into the environment of use. The water-swellable composition comprises a swelling agent in an amount ranging from about 30 to 100 wt % of the water-swellable composition. The swelling agent is generally a water-swellable polymer that greatly expands in the presence of water.

Suitable swelling agents for the water-swellable composition are generally hydrophilic polymers. Exemplary hydrophilic polymers include polyoxomers such as PEO, cellulosics such as HPMC and HEC, and ionic polymers. In general, the molecular weight of water swellable polymers chosen for the swelling agent is higher than that of similar polymers used as entraining agents (see above) such that, at a given time during drug release, the water-swellable composition after imbibing water tends to be more viscous, less fluid, and more elastic relative to the drug-containing composition. In some cases the swelling agent may be even substantially or almost entirely water insoluble such that when partially water swollen during operation, it may constitute a mass of water-swollen elastic particles. Generally, the swelling agent is chosen such that, during operation, the water-swellable composition generally does not substantially intermix with the drug-containing composition, at least prior to extruding a majority of the drug-containing composition.

The water-swellable composition may optionally include osmotically-effective solutes, tableting aids, solubility-enhancing agents or excipients that promote stability, or processing of the dosage form of the same types mentioned above.

Diffusion Devices

In another embodiment, the controlled-release dosage form has two components: (a) a core containing the drug; and (b) an asymmetyric non-dissolving and non-eroding coating surrounding the core, the coating controlling the rate at which drug diffuses out of the core into the environment of use. Thicker coatings or coatings having lower porosity generally have slower release rates. Also, coatings with lower drug permeability generally have slower release rates, particularly non-porous coatings. Diffusion devices are described in the following U.S. Pat. Nos. 4,186,184 and 5,505,962.

The core comprises the drug and other excipients, such as tableting aids, surfactants, water-soluble polymers, pH modifiers, fillers, binders, pigments, disintegrants, antioxidants, lubricants, flavorants, and solubility-enhancing agents as described above.

The Coating

All of the controlled-release dosage forms described above comprise a drug-containing core and an asymmetric coating. The asymmetric coating controls the rate at which drug is released to the use environment either by controlling the transport of water from the use environment to the core, or by controlling the diffusion of drug out of the core to the use environment. The inventors have found that in order for the rate of drug release to be the same in a use environment containing a substantial amount of dietary fat (or dietary fat digestion products) compared to the rate of drug release in a use environment that does not contain a substantial amount of dietary fat, the materials used for making the asymmetric coating must be carefully selected.

Asymmetric coatings are known to the art, for example as disclosed in U.S. Pat. No. 5,612,059 to Cardinal et al. Such coatings are membranes that consist of a very thin, dense skin supported by a thicker, porous substructure layer. Delivery devices that can be made with asymmetric membranes include tablets, capsules, and beads. Such membranes can be made by a phase inversion process, as disclosed in the aforementioned patent. Advantageously, and as also disclosed therein, the porosity of the membrane can be implemented in a controlled manner such that the porosity, and hence the rate of release, can be tailored. By tailoring the rate of release, the release profile of the resulting delivery composition can be controlled and tailored as well.

The inventors have observed that drug release from dosage forms with asymmetric polymeric membrane coatings demonstrates that some of the coating polymers, but not all, while successfully demonstrating desirable release characteristics when administered under fasted conditions, can exhibit significant reduction of drug release if administered following a high fat meal.

It has been found that such changes in performance of the dosage forms can be attributed to swelling of the asymmetric membrane polymer by fats and fat digestion products present in the high-fat use environment. This characteristic could also cause rapid release, or dose dumping, in some dosage forms.

To avoid such effects, it has been found that the asymmetric membrane polymer used to form the coating around the core should swell less than about 15 wt %, preferably less than about 5 wt % when soaked for at least 16 hours in an aqueous solution of 0.5 wt % hydrolyzed dietary fat mixture. An example of a suitable hydrolyzed dietary fat mixture is the 50% hydrolyzed model oil, previously described. Generally, the water permeability of materials that swell more than this changes significantly when placed into a use environment containing a substantial amount of dietary fat (or dietary fat digestion products), leading to a change in the rate of controlled release of the drug from the core.

The following procedure can be used to screen polymers for use in making asymmetric membranes for dosage forms is as follows. Dense films of polymers (e.g., 10 μm to 200 μm thickness) can be made, for example, by dissolving the candidate polymer in an appropriate solvent and casting this polymer solution onto an appropriate surface (e.g., a glass plate) using, for example, a Gardner casting knife (Gardner Labs, Inc., Bethesda, Md.). Any volatile solvent for the polymer to be screened may be used, as well as any casting technique that produces a dense film. The films can be air-dried to allow evaporation of solvent and the resulting film removed from the casting surface. Small pieces of the dense film (e.g., 10 to 20 mg dry weight) are first placed into 0.01 M HCl solution stirred at 50 rpm at 37° C. for at least 3 hours. Each piece of dense film is then removed from the solution, patted dry with absorbent paper to remove surface water, and weighed. The pieces of dense film are then placed into a use environment consisting of 0.5 wt % 50% hydrolyzed model oil in simulated gastric buffer comprising 0.01 M HCl at 37° C. and agitated at 50 rpm for 21 to 48 hours. The films are then removed, patted dry with absorbent paper to remove surface water, and weighed. The amount of material absorbed into the dense film is then calculated by the following equation:

$$Amount absorbed ( wt \% ) = ( \frac{Weight after soaking in the use environment}{Weight after soaking in 0.01 \underline{M} HCl solution} 1 ) * 100$$

Examples of suitable coating materials include cellulose acetate, cellulose acetate butyrate, cellulose acetate proprionate, cellulose acetate phthalate, hydroxypropyl methyl cellulose acetate succinate, polymethacrylate, and mixtures and blends thereof. A preferred coating material is cellulose acetate. By “cellulose acetate” is meant a family of cellulosic polymers that have acetate groups attached via ester linkages to a portion of the cellulosic polymer's hydroxyl groups. The degree of substitution of acetate on the cellulosic polymer can range from 0.1 to 3. “Degree of substitution” refers to the average number of the three hydroxyls per saccharide repeat unit on the cellulose chain that have been substituted. Also included are cellulose acetates that have additional substituents added in relatively small amounts that do not substantially alter the performance of the polymer. The molecular weight of the cellulose acetate should be sufficiently high to provide a high-strength coating, but low enough for readily processing the material during the coating process. Preferably, the cellulose acetate has an average molecular weight of greater than about 10,000 daltons, but less than about 100,000 daltons. More preferably, the cellulose acetate has an average molecular weight of greater than 25,000 daltons but less than about 75,000 daltons. A preferred polymer is cellulose acetate having an acetyl content of 39.8%, and specifically, CA 398-10 manufactured by Eastman of Kingsport, Tenn., having an average molecular weight of about 40,000 daltons. Another preferred polymer having an acetyl content of 39.8% is CA-398-30 (Eastman) reported to have an average molecular weight of 50,000 daltons.

The coating may be applied to the core in a manner that is conventional, but which makes it asymmetric, for example by first forming a coating solution, coating it onto cores by dipping, fluidized bed coating, or pan coating, and by then inducing the solution to undergo phase separation in a particular way, resulting in a structured, continuous polymer phase. To accomplish this, a coating solution is formed comprising the coating polymer or polymers and a solvent. Typical solvents include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. A particularly preferred solvent is acetone. The coating solution typically will contain 3 to 15 wt % of the polymer, preferably 5 to 12 wt %, most preferably 7 to 12 wt %. The coating solution is coated on the core of a delivery device, such as a tablet core, and, then dried, forming the structured membrane on the core.

Generally, the outside surface of the asymmetric coating is a skin that will have a higher density than the coating nearest the core. As disclosed above, the asymmetric coating may be formed by a phase inversion process in which the coating polymer is dissolved in a mixture of solvents and non-solvents chosen such that as the coating dries, a phase inversion takes place in the applied coating solution, resulting in the formation of a porous solid with a thin dense outer region. This type of membrane, similar to those used in the reverse-osmosis industry, generally allows higher osmotic fluxes of water than can be obtained with a dense membrane.

The coating solution may also comprise pore-formers, non-solvents, other polymers or mixtures of polymers (described more fully below), or plasticizers in any amount so long as the polymer remains substantially soluble at the conditions used to form the coating and so long as the coating remains permeable, and asymmetric, and does not significantly change permeability when placed into a use environment containing a high concentration of dietary fats. The term “pore former,” as used herein, refers to a material added to the coating solution that has low or no volatility relative to the solvent such that it remains as part of the coating following the coating process but that is sufficiently water swellable or water soluble such that, in the aqueous use environment it provides a water-filled or water-swollen channel or “pore” to allow the passage of water thereby enhancing the water permeability of the coating. Suitable pore-formers include polyethylene glycol (PEG), PVP, PEO, HEC, HPMC and other aqueous-soluble cellulosics, water-soluble acrylate or methacrylate esters, polyacrylic acid and various copolymers and mixtures of these water soluble or water swellable polymers. Enteric polymers such as cellulose acetate phthalate (CAP) and HPMCAS are included in this class of polymers. The pore former can also be a sugar, organic acid, or salt. Examples of suitable sugars include sucrose and lactose; examples of organic acids include citric and succinic acid; examples of salts include sodium chloride and sodium acetate. Mixtures of such compounds may also be used.

For the formation of porous coatings, a non-solvent may be added to the coating solution. By “non-solvent” is meant any material added to the coating solution that substantially dissolves in the coating solution and reduces the solubility of the coating polymer or polymers in the solvent. In general, the function of the non-solvent is to impart porosity to the resulting coating. The preferred non-solvent depends on the solvent and the coating polymer chosen. In the case of using a volatile polar coating solvent such as acetone or methyl ethyl ketone, suitable non-solvents include water, glycerol, ethylene glycol and its low molecular-weight oligomers (e.g., less than about 1,000 daltons), propylene glycol and its low molecular weight oligomers (e.g., less than about 1,000 daltons), C₁ to C₄ alcohols such as methanol or ethanol, ethylacetate, acetonitrile and the like.

The coating can optionally include a plasticizer. A plasticizer generally swells the coating polymer such that the polymer's glass transition temperature is lowered, its flexibility and toughness increased and its permeability altered. When the plasticizer is hydrophilic, such as polyethylene glycol, the water permeability of the coating is generally increased. When the plasticizer is hydrophobic, such as diethyl phthalate or dibutyl sebacate, the water permeability of the coating is generally decreased.

The coating can optionally include other polymers. For example, water soluble polymers may be included as pore-formers. Alternatively high strength polymers could be included to increase durability of the coating.

For delivery device cores which release drug primarily by extrusion, the asymmetric coating must also contain at least one delivery port in communication with the interior and exterior of the coating to allow for release of the drug-containing composition to the exterior of the dosage form. The delivery port can range in size from about the size of the drug particles, and thus could be as small as 1 to 100 microns in diameter and may be termed pores, up to about 5000 microns in diameter. The shape of the port may be substantially circular, in the form of a slit, or other convenient shape to ease manufacturing and processing. The port(s) may be formed by mechanical or thermal means or with a beam of light (e.g., a laser), a beam of particles, or other high-energy source (see, for example, U.S. Pat. Nos. 5,783,793, 5,658,474, 5,399,828, 5,376,771, and 5,294,770), or may be formed in situ by rupture of a small portion of the coating (see, for example, U.S. Pat. Nos. 5,736,159, 5,558,879, and 4,016,880). Such rupture may be controlled by intentionally incorporating a relatively small weak portion into the coating. Delivery ports may also be formed in situ by erosion of a plug of water-soluble material or by rupture of a thinner portion of the coating over an indentation in the core. Delivery ports may be formed by coating the core such that one or more small regions remains uncoated. In addition, the delivery port can be a large number of holes or pores that may be formed during coating, as in the case of porous membrane coatings of the type disclosed in U.S. Pat. Nos. 5,612,059 and 5,698,220. When the delivery pathways are pores there can be a multitude of such pores that range in size from 1 μm to greater than 100 μm. During passage through the GI tract, one or more of such pores may enlarge under the influence of the hydrostatic pressure generated during operation. The number of delivery ports may vary from 1 to 10 or more. For delivery device cores which consist of separate drug and sweller layers, at least one delivery port should be formed on the side of the coating that is adjacent to the drug-containing composition, so that the drug-containing composition will be extruded out of the delivery port by the swelling action of the water-swellable composition. It is recognized that some processes for forming delivery ports may also form holes or pores in the coating adjacent to the water-swellable composition. In aggregate, the total surface area of core exposed by delivery ports is less than 5%, and more typically less than 1%.

Once a controlled-release composition (e.g., a core surrounded by an asymmetric rate-limiting membrane) has been formed, one or more additional coatings may be applied as further outside coatings, usually on top of and usually surrounding a rate-limiting membrane. The additional coatings typically comprise materials that are soluble in the environment of use and the materials should not be affected by the presence of fat in the use environment, as previously described. When applied to the composition, the additional coating(s) should not affect the water permeability or morphology (e.g., porosity, pore size) of the rate-limiting coating.

Such coatings can be used for a variety of purposes well known in the arts, including (1) to mask the taste or odor of the composition, (2) to provide physical and chemical protection for the composition, and (3) to improve the appearance of the composition, such as through use of special colors and contrasting printing. See for example, The Theory and Practice of Industrial Pharmacy, by Lachman, Lieberman, and Kanig (3^{rd} Edition, 1986, Lea & Febiger, Philadelphia).

An additional coating may also be applied to the composition that provides an immediate release of the active present in the core, or an immediate release of a second active. When administered to an aqueous environment of use, the immediate-release coating supplies an immediate release of drug in addition to the drug that is released in a controlled release manner from the core of the composition.

As previously discussed, the compositions of this invention can be administered to human patients or subjects who have imbibed a high fat meal and have, in essence, thereby converted their gastrointestinal tract into an in vivo high fat use environment. To this end, and as an additional feature of the invention, this invention provides a therapeutic package suitable for commercial sale, comprising a container, an oral dosage form of a therapeutic agent contained therein which is in a core/asymmetric membrane controlled release delivery composition according to the invention and, associated with said package, written (i.e., printed) matter non-limited as to whether the dosage form can be taken with or without food of any type, particularly food which effects, in vivo, a high fat environment. Although Applicants do not wish to limit the nature of the written matter, it is noted that the written matter is generally of the type containing labeling, i.e., information and/or instructions for the physician, pharmacist or patient, including language of the type which a regulatory agency (such as the US Food and Drug Administration) directs or permits the package labeling or insert to contain. The written material can be non-limited by virtue of containing no statement regarding whether or not the dosage form can be taken with or without food, high fat or otherwise, i.e., by virtue of being silent. Alternatively, the written material can contain one or more non-limiting statements affirmatively informing the user (i.e., the patient, pharmacist, and/or physician) that the oral dosage form can be taken by or administered to a patient regardless of whether the patient has eaten or imbibed high fat food, or a statement such as “may be taken without regard to type or quantity of food” or something similar such as “may be taken without regard to the quantity of fat in food”. The written language can not contain limiting language such as “This dosage form may not be taken with a high fat meal” or “This dosage form should be administered at least one hour before or at least two hours after eating”, or similar language imparting the same or a similar message.

The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual dosages for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

The printed or otherwise written matter is associated with the package in which the therapeutic dosage form is sold. The term “associated with” is intended to include all manners in which written matter, such as the instructional or informational materials discussed above, i.e., labeling, can be associated with a medicament, as known conventionally in the art. Thus written matter can be associated with the container, for example, by being: written on a label (e.g., the prescription label or a separate label) adhesively affixed to a bottle containing a quantity of therapeutic dosages; included inside a container such as a box or bottle as a written package insert, for example inside a box which contains a bottle of tablets; applied directly to the container such as being printed on the wall of the box; or attached as by being tied or taped, for example as an instructional card affixed to the neck of a bottle via a string, cord or other line, lanyard or tether type device. The written matter may be printed directly on a box or blister pack or blister card. The written matter may (and usually will) contain other information (usually regulatory information) in addition to, if one is included, a statement informing that the dosage forms may be taken with high fat food.

Other features and embodiments of the invention will become apparent from the following examples which are given for illustration of the invention rather than for limiting its intended scope. In the examples, the following definitions are employed: mgA—milligrams of active drug having a molecular weight determined as the free acid or base, independent of salt form; CFM—cubic feet per minute; RPM—revolutions per minute; AUC—Area under the concentration versus time curve determined in blood or plasma; CA—cellulose acetate; CAB—Cellulose acetate butyrate; CAP—cellulose acetate phthalate;

### EXAMPLES

### Example 1

Several polymers intended for testing to determine their suitability as asymmetric membrane coating materials for a wide variety of dosage forms of the present invention were examined for their suitability for use in a high-fat environment. GI fluid following ingestion of a high-fat meal was simulated by a mixture of 0.5 wt % “50% hydrolyzed model oil” mixed in a 0.01M HCl aqueous solution. Polymers were either obtained as commercial films or formed into dense films by casting a polymer solution onto a glass plate using a Gardner knife (Gardner labs, Inc., Bethesda, Md.). Table I lists the polymers tested, the polymer solution composition used for casting films and the final thickness of each type of film. Following casting, solvent was allowed to evaporate overnight at ambient conditions (22° C.). Films were then soaked in water for 30 seconds to 5 minutes, removed from the glass plate, and then dried in a 37° C. oven for at least 16 hours to remove all of the coating solvent prior to evaluation.

Individual pieces of polymer film ranging in size from 5 to 30 cm² and 20 to 70 mg in weight were first weighed and then placed in 19.9 mL 0.01 M HCl stirred at 37° C. in a glass vial for at least 3 hours to equilibrate with the aqueous solution. Each film was then removed, patted dry with absorbent paper and weighed. Next, 0.1 gram of the “50% hydrolyzed model oil” was added to the 0.01 M HCl solution in each vial and the films replaced. The films remained in the solutions, which were stirred at 37° C. for 21 to 48 hours and then removed, wiped dry with absorbent paper and weighed. The average weight increase for three replicates of each film type between dry conditions and after soaking in 0.01 M HCl, and between 0.01 M HCl and the 0.5 wt % “50% hydrolyzed model oil”, is given in Table II. These results show that films composed of polymers Number 1 through Number 11 showed weight increase from contact with the “50% hydrolyzed model oil” of 15 wt % or less and are, therefore, suitable polymers for use in this invention. Polymers 12 through 14 showed weight increases from contact with the “50% hydrolyzed model oil” of more than 34 wt % and are, correspondingly, unsuitable for use in the invention.

TABLE I

| | | | | Film Preparation | | |
|---|---|---|---|---|---|---|
| | Polymer | | | | Polymer | Film |
| | Commercial | Polymer | | | Conc. | Thickness |
| No. | Name | Type | Manufacturer | Solvent | (wt %) | (μm) |
| 1 | CA-398-10 | Cellulose | Eastman | Acetone | 10% | 109 |
| | NF | Acetate, | Chemical Co. | | | |
| | | Acetate | | | | |
| | | content = 39.8% | | | | |
| 2 | CA-435- | Cellulose | FMC Corp. | MeCl₂ | 10% | 97 |
| | 75S | Acetate, | Food and | | | |
| | | Acetate | Pharmaceutical | | | |
| | | content = 43.3-43.9% | Products | | | |
| | | | Div. | | | |
| 3 | CA320S | Cellulose | Eastman | 90:10 | 8.20% | 102 |
| | | Acetate, | Chemical Co. | MeCl₂/ | | |
| | | Acetate | | MeOH | | |
| | | content = 39.8% | | | | |
| 4 | Cellulose, | Regenerated | BCL Canada | Commercial Film | | 30 |
| | PUVT 214 | Cellulose | Inc. | | | |
| | | film | | | | |
| 5 | CAB-551-0.2 | Cellulose | Eastman | Acetone | 23% | 130 |
| | | Acetate | Chemical Co. | | | |
| | | Butyrate, | | | | |
| | | Acetate | | | | |
| | | content = 2.0% | | | | |
| 6 | CAB-381- | Cellulose | Eastman | Acetone | 15% | 102 |
| | 20 | Acetate | Chemical Co. | | | |
| | | Butyrate, | | | | |
| | | Acetate | | | | |
| | | content = 13.5% | | | | |
| 7 | CAB-171- | Cellulose | Eastman | Acetone | 14% | 91 |
| | 15 | Acetate | Chemical Co. | | | |
| | | Butyrate, | | | | |
| | | Acetate | | | | |
| | | content = 29.9% | | | | |
| 8 | CAP 482- | Cellulose | Eastman | Acetone | 19% | 107 |
| | 20 | Acetate | Chemical Co. | | | |
| | | Propionate, | | | | |
| | | Acetate | | | | |
| | | content = 1.5% | | | | |
| 9 | C-A-P | Cellulose | Eastman | Acetone | 21% | 94 |
| | | Acetate | Chemical Co. | | | |
| | | Phthalate | | | | |
| | | NF | | | | |
| 10 | HPMCAS | Hydroxypropyl | Shin-Etsu | Acetone | 17% | 102 |
| | AS-HF | Methylcellulose | Chemical | | | |
| | | Acetate | Co., Ltd. | | | |
| | | Succinate | | | | |
| 11 | Eudragit | Polymethacrylate | Rohm & | Acetone | 33% | 178 |
| | RS100 | | Haas | | | |
| 12 | EVAL EF-F | Ethylene/Vinyl | EVAL | Commercial Film | | 13 |
| | | Alcohol | Company of | | | |
| | | Copolymer | America | | | |
| 13 | Shellac | Shellac | Spectrum | Acetone | 41% | 135 |
| | Gum, | Gum | Quality | | | |
| | refined | | Products, | | | |
| | | | Inc. | | | |
| 14 | Ethocel | Ethylcellulose | The Dow | Acetone | 11% | 89 |
| | S100 | NF | Chemical Co. | | | |
| | | Premium | | | | |

TABLE II

| | | Average Percent | Average Percent |
|---|---|---|---|
| | | Weight Gain | Weight Gain (0.01 M |
| Polymer | | (dry to 0.01 | HCl to 0.5 wt % “50% |
| No. | Polymer Type | M HCl)** | hydrolyzed model oil”) |
| 1 | Cellulose Acetate, | 9.7 | 2.7 |
| | Acetate content = | | |
| | 39.8% | | |
| 2 | Cellulose Acetate, | 8.7 | 1.0 |
| | Acetate content = | | |
| | 43.3-43.9% | | |
| 3 | Cellulose Acetate, | 27.3 | −0.7 |
| | Acetate content = | | |
| | 39.8% | | |
| 4 | Regenerated | 93.3 | 0.3 |
| | Cellulose film | | |
| 5 | Cellulose Acetate | 2.0 | 5.0 |
| | Butyrate, | | |
| | Acetate | | |
| | content = 2.0% | | |
| 6 | Cellulose Acetate | 2.3 | 2.3 |
| | Butyrate, | | |
| | Acetate | | |
| | content = 13.5% | | |
| 7 | Cellulose Acetate | 5.3 | 1.0 |
| | Butyrate, | | |
| | Acetate | | |
| | content = 29.9% | | |
| 8 | Cellulose Acetate | 4.0 | 2.7 |
| | Propionate, | | |
| | Acetate | | |
| | content = 1.5% | | |
| 9 | Cellulose Acetate | 9.7 | −0.3 |
| | Phthalate NF | | |
| 10 | Hydroxypropyl | 12.3 | 1.3 |
| | Methylcellulose | | |
| | Acetate Succinate | | |
| 11 | Polymethacrylate | 15.3 | 14.3 |
| 12 | Ethylene/Vinyl | 9.7 | 34.0 |
| | Alcohol Copolymer | | |
| 13 | Shellac Gum | 4.7 | 36.0 |
| 14 | Ethylcellulose NF | 4.0 | 43.0 |
| | Premium | | |

**Average of three films. Average Percent Weight Gain = [(Final weight − initial weight)/Initial weight] × 100

### Example 2.

Polymers used as coating materials to make asymmetric membranes in a wide variety of dosage forms of the present invention were cast into films as described in Example 1. The films were exposed to individual components of dietary fat mixtures and model mixtures simulating a use environment containing a substantial amount of dietary fat and/or dietary fat digestion products. Dense films of the materials were cast from acetone solutions. Three grades of ethylcellulose (Ethocel® S100, Ethocel M70, and Ethocel M50) and one grade of cellulose acetate (CA398-10), were examined. Films of polymer blends (Ethocel S100 and CA398-10) were also used. Small pieces of the resultant films (10-20 mg dry weight) were placed in 0.05% MFD containing 3 wt % of fat components being tested. The solutions were shaken at 37° C. for at least 20 hours. The film pieces were recovered, wiped clean, and weighed.

The results are tabulated below in Tables III and IV; the formulations used in the mixtures are given in Table V. As shown in Table III, all three grades of Ethocel were swollen by carboxylic acids, by many monoglycerides, and by triglycerides (e.g., tributyrin). The Ethocel materials also showed significant swelling in the mixtures of these compounds. These materials effected, when swollen, weight gains generally in excess of 20 wt %.

The data in Table III show that the cellulose acetate material showed little weight gain or swelling in all compounds tested, indicating cellulose acetate will be an excellent choice for use as a coating material that does not change in the presence of substantial amounts of dietary fat or dietary fat digestion products.

The data in Table IV show that the polymer blends also swelled considerably when exposed to the fat components evaluated.

These data indicate that swelling of the materials based on Ethocel is caused primarily by compounds produced by hydrolysis of the fat: fatty acids and monoglycerides.

TABLE III

| | | Weight Gain (wt %) | | | |
|---|---|---|---|---|---|
| Test solution | | Ethocel | Ethocel | Ethocel | CA398- |
| Class | Material | S100 | M70 | M50 | 10 |
| Carboxylic Acids | Butyric Acid | 28 | ND* | 25 | 16 |
| | Decanoic | 140 | ND | ND | ND |
| | Acid | | | | |
| | Oleic Acid | 77 | 410 | 190 | 10 |
| Monoglycerides | Imwitor 375 | 10 | 10 | ND | ND |
| | Monoolein | 12 | ND | ND | ND |
| | Imwitor 312 | 13 | 13 | ND | ND |
| | Monolineolin | 24 | ND | ND | ND |
| | Capmul MCM | 96 | ND | ND | ND |
| | Monocaprylin | 110 | 120 | 85 | 18 |
| | Monobutyrin | 130 | ND | 55 | 22 |
| | Imwitor 742 | 230 | 230 | 220 | 15 |
| Triglycerides | Triacetin | 11 | ND | ND | ND |
| | Tricaprylin | 71 | ND | 67 | 18 |
| | Tributyrin | 340 | ND | 260 | 17 |
| Mixtures** | Model Oil | 50 | 8.3 | 100 | 6.1 |
| | Model Oil | >500 | ND | ND | 8 |
| | Products A | | | | |
| | Model Oil | 530 | ND | ND | 7 |
| | Products C | | | | |
| | 50% | 600 | 47 | 360 | 4.3 |
| | Hydrolyzed | | | | |
| | Model Oil | | | | |
| | Model Oil | 800 | ND | ND | 7 |
| | Products B | | | | |

*ND = not determined
**See Table V

TABLE IV

| | | Weight Gain (wt %)* | | | | |
|---|---|---|---|---|---|---|
| Test solution | | 95/5 | 90/10 | 80/20 | 60/40 | 30/70 |
| Class | Material | Blend | Blend | Blend | Blend | Blend |
| Carboxylic | Butyric Acid | 47 | 29 | 33 | 30 | 22 |
| Acids | Caprylic | 91 | 150 | Dis- | 96 | 210 |
| | acid | | | solved | | |
| | Oleic Acid | 280 | 190 | 260 | 170 | 12 |
| Monoglycerides | Monocaprylin | 80 | 77 | 70 | 86 | 39 |
| | Monobutyrin | 50 | 48 | 42 | 42 | 58 |
| | Imwitor 742 | 200 | 210 | 230 | 88 | 33 |
| Triglycerides | Tricaprylin | 75 | 160 | 110 | 78 | 26 |
| | Tributyrin | 120 | 190 | 250 | 190 | 58 |
| Mixtures** | Model Oil | 15 | 30 | 23 | 31 | 15 |
| | 50% | 270 | 220 | 180 | 150 | 20 |
| | Hydrolyzed | | | | | |
| | Model Oil | | | | | |

*Weight ratio of Ethocel S100/CA398-10 in the blend
**See Table V

TABLE V

| Oil | Components |
|---|---|
| Model Oil | 75% olive oil, 18% tripalmitin, 6% tributyrin, 1% lecithin |
| 50% | 37% olive oil, 15% Myverol 18-99, 23% oleic acid, |
| Hydrolyzed | 9% tripalmitin, 4% Imwitor 191, 5% palmitic |
| Model Oil | acid, 3% tributyrin, 2% butyric acid, |
| | 1% monobutyrin, 1% lecithin |
| Model Oil | 42% oleic acid, 20% Myverol 18-99, 8% Myverol 18-92, |
| Products A | 7% im191, 9% palmitic acid, 6% tributyrin, 4% butyric |
| | acid, 2% monobutyrin, 2% lecithin |
| Model Oil | 51% oleic acid, 20% Myverol 18-99, |
| Products B | 15% Myverol 18-92, 6% tributyrin, |
| | 4% butyric acid, 2% monobutyrin, 2% lecithin |
| Model Oil | 51% oleic acid, 20% Myverol 18-99, |
| Products C | 15% Myverol 18-92, 6% tributyrin, |
| | 5% butyric acid, 3% monobutyrin |

### Example 3

Controlled release tablets containing pseudoephedrine and coated with ethylcellulose were manufactured as follows. First, a blend was prepared containing 75.4 wt % pseudoephedrine HCl, 3.4 wt % hydroxypropyl cellulose and 21.2 wt % microcrystalline cellulose. The blend was wet granulated in a P-K processor and dried. The dried granulation was milled using a Fitzpatrick mill, then mixed in a V-blender. The dried granulation (59.8 wt %) was blended with microcrystalline cellulose (40.2 wt %), milled using a Fitzpatrick mill, and blended again. The final blend was prepared by adding 0.5 wt % magnesium stearate and mixing. Tablets containing 240 mg of pseudoephedrine HCl were made from this blend on a rotary tablet press using 7/16″ tooling and a target tablet weight of 537 mg.

The cores were then coated with an asymmetric ethylcellulose membrane formed by the phase-inversion process disclosed in U.S. Pat. Nos. 5,612,059 and 5,698,220 as follows. A solution containing 82.3 wt % acetone, 7.7 wt % water, 3.4 wt % polyethylene glycol 3350 and 6.6 wt % of ethylcellulose (Ethocel standard 100 premium) was prepared by mixing these ingredients in a solution make-up tank. The coating solution was applied to the tablet cores in a perforated coating pan (HCT-60, Vector Corporation) using one spray gun, a spray rate of 210 mL/min, an inlet air temperature of 48° C., an inlet air volume of 300 CFM, and a pan speed of 15 RPM resulting in an asymmetric coating on the tablet cores. A target weight gain of 99 mg was achieved during coating. The coated tablets were dried in a tray dryer.

These asymmetric ethylcellulose-coated tablets were then coated with an immediate-release layer of a second drug, cetirizine. For the cetirizine coating, an aqueous solution of 2 wt % cetirizine HCI and 3.9 wt % clear Opadry® YS-5-19010 Clear (major components include hydroxypropyl cellulose and hydroxypropyl methycellulose), Colorcon, West Point, Pa. was prepared and mixed for 2 hours. The cetirizine-containing layer was applied to the ethylcellulose coated tablets in a perforated coating pan (HCT-60, Vector Corporation) using two spray guns, a solution spray rate of 40 g/min, an inlet air temperature of 74° C., an inlet air volume of 280 CFM and a pan speed of 16 RPM. Enough solution was sprayed until 10 mgA of drug was applied to each tablet.

The immediate-release cetirizine layer was then coated with a taste-mask coating. For the taste-mask coating,10 wt % White Opadry® YS-5-18011 White (major components include hydroxypropyl cellulose and hydroxypropyl methycellulose), Colorcon, West Point, Pa. was added to water and mixed for 2 hours. This coating solution was applied to the tablets in a perforated coating pan (HCT-60, Vector Corporation) using one spray gun, an inlet air temperature of 84° C., an inlet air volume of 300 CFM, a solution spray rate of 60 g/min, and a pan speed of 16 RPM. Enough solution was sprayed until 20 mg of coating was applied to each tablet.

### Example 4

Controlled release tablets containing pseudoephedrine and coated with an asymmetric cellulose acetate coating were manufactured as follows. First, a blend was prepared containing 75.4 wt % pseudoephedrine HCl, 3.4 wt % hydroxypropyl cellulose and 21.2 wt % microcrystalline cellulose, and processed as described in Example 3. Tablets containing 240 mg of pseudoephedrine HCl were made from this blend on a rotary tablet press using 7/16″ tooling and a target tablet weight of 543 mg.

Next, the cores were coated with a porous asymmetric cellulose acetate membrane, made as disclosed in U.S. Pat. Nos. 5,612,059 and 5,698,220 as follows. A solution containing 70.2 wt % acetone, 18 wt % water, 2.6 wt % polyethylene glycol 3350 and 9.2 wt % of cellulose acetate 398-10 was prepared by mixing these ingredients in a solution make-up tank. The coating solution was applied to the tablet cores in a perforated coating pan (HCT-60, Vector Corporation) using one spray gun, a spray rate of 135 mL/min, an inlet air temperature of 45° C., an inlet air volume of 300 CFM, and a pan speed of 14 RPM, resulting in the formation of an asymmetric coating on the tablet cores. A target weight gain of 92 mg was achieved during coating. The coated tablets were dried in a tray dryer.

### Example 5

Sunepitron tablets coated with ethylcellulose were manufactured as follows. First, a blend was prepared containing 3.7 wt % sunepitron, 8.3 wt % fumaric acid and 87.5 wt % anhydrous lactose in a high shear mixer. Next, 0.25 wt % magnesium stearate was added and a dry granulation was produced with a roller compactor. The ribbons were milled through an oscillating granulator and blended in a V-blender. The final blend was prepared by adding 0.25 wt % magnesium stearate and mixing. Tablets containing 10 mg of sunepitron were made from the blend on a rotary tablet press using 11/32″ standard round concave tooling at a target tablet weight of 300 mg.

Next, the cores were coated with a porous asymmetric ethylcellulose membrane as follows. A solution containing 53.2 wt % acetone, 10.9 wt % isopropanol, 22.4 wt % ethanol, 3.0 wt % water, 4.5 wt % polyethylene glycol 3350 and 6.0 wt % ethylcellulose (Ethocel standard 100 premium) was prepared by mixing these ingredients in a stainless steel vessel. The coating solution was applied to the tablet cores in a perforated coating pan (an HCT-30, Vector Corporation) using one spray gun, a solution spray rate of 32 g/min, an outlet air temperature of 25° C., and inlet air volume of 40 CFM, and a pan speed of 25 RPM, resulting in the formation of an asymmetric coating on the tablet cores. A target weight gain of 60 mg was achieved during coating. The coated tablets were dried overnight in a tray dryer.

### Example 6

Sunepitron tablets coated with an asymmetric cellulose acetate membrane were manufactured as follows. First, a blend was prepared containing 3.7 wt % Sunepitron, 8.3 wt % fumaric acid and 86.0 wt % anhydrous lactose using the procedure outlined in Example 5. Next, 1.0 wt % magnesium stearate was added and a dry granulation was produced with a roller compactor. The ribbons were milled (Fitzpatrick JT mill) and blended in a V-blender. The final blend was prepared by adding 1.0 wt % magnesium stearate and mixed. Tablets containing 10 mg of the drug substance were made from the blend on a rotary tablet press using 11/32″ extra deep round concave tooling at a target tablet weight of 300 mg. Next, the cores were coated with a porous asymmetric cellulose acetate membrane as follows. A solution containing 52.9 wt % acetone, 10.5 wt % isopropanol, 22.0 wt % ethanol, 2.6 wt % water, 4.0 wt % glycerol and 8.0 wt % cellulose acetate (398-10) was prepared by mixing these ingredients in a stainless steel vessel. The coating solution was applied to the tablet cores in a perforated coating pan (an HCT-30 Vector Corporation) using one spray gun, a solution spray rate of 32 g/min, an outlet air temperature of 25° C., an inlet air volume of 40 CFM, and a pan speed of 25 RPM, resulting in the formation of an asymmetric membrane on the tablet cores. A target weight gain of 45 mg was achieved during coating. The coated tablets were dried overnight in a tray dryer.

### Example 7

The pseudoephedrine-containing tablets of Examples 3 and 4 were dissolution tested as follows. Tablets were tested in 1000 mL deionized water (the control test media), or in 500 mL standard blended breakfast mixed with simulated intestinal fluid containing enzymes (SBB/SIF). The SIF was prepared as follows. First, 6.8 g of monobasic potassium phosphate was dissolved in 250 mL of water. Next, 190 mL of 0.2 N sodium hydroxide was mixed with 400 mL of water and combined with the potassium phosphate solution. Next, 10 g of pancreatin was added, and the pH of the resulting solution was adjusted to 7.5±0.1 with 0.2 N sodium hydroxide. Water was added for a final volume of 1000 mL.

The SBB/SIF was prepared as follows. To 250 mL of SIF was added

2 pieces of white toast with butter

2 strips of bacon

6 oz of hashbrowns

2 eggs scrambled in butter

8 oz of whole milk or about 250 mL

8 g of extra butter

This solution was mixed in an industrial single speed Waring Blender.

For dissolution tests using deionized water, pseudoephedrine release was measured by directly analyzing its concentration in the 1000 mL deionized water receptor solution as a function of time. The receptor solution, in a dissolution apparatus (Hanson Dissoette™ Autosampler, Hanson Research Corporation, Chatsworth, Calif.) fitted with standard paddles, was stirred at 75 rpm and held at 37° C. For dissolution tests using SBB/SIF, pseudoephedrine released was measured by residual analysis of tablets that were in the receptor solution for the specified times. The receptor solution, in a standard dissolution apparatus (USP Type II, VanKel, Cary, N.C.) fitted with standard paddles, was stirred at 75 rpm and held at 37° C. In both cases, pseudoephedrine concentrations were measured using an HPLC method using a Zorbax Stablebond® CN column with a mobile phase of 50% 0.1 M KH₂PO₄, pH 6.5/50% methanol containing 1 g/L sodium octanesulfonate, and UV detection at 214 nm.

The results of the tests, summarized in Table VI, show that the amount of pseudoephedrine released from the tablets coated with cellulose acetate tested in a high-fat use environment (the SBB/SIF solution) ranged from 1.0-fold to 1.6-fold that of the same tablets evaluated in a use environment that does not contain a substantial amount of dietary fat (distilled water) between 2 and 6 hours after introduction into the use environment. However, the tablets coated with Ethocel showed extremely slow release, with the amount of pseudoephedrine released from the tablets coated with Ethocel tested in a high-fat use environment (the SBB/SIF solution) ranging from 0.3-fold to 0.04-fold that of the same tablets evaluated in a use environment that does not contain a substantial amount of dietary fat (distilled water) between 2 and 6 hours after introduction into the use environment.

TABLE VI

| | Elapsed | Pseudoephedrine | | Ratio |
|---|---|---|---|---|
| | Time | Released % | | “SBB/SIF”/Distilled |
| Example | (hr) | Distilled Water | SBB/SIF | Water |
| CA Coated | 0 | 0 | 0 | NA* |
| Tablets | 1 | 0 | 2 | NA |
| | 2 | 3 | 3 | 1.0 |
| | 4 | 15 | 22 | 1.5 |
| | 6 | 29 | 46 | 1.6 |
| Ethocel | 0 | 0 | 0 | NA |
| Coated | 1 | 1 | 2 | NA |
| Tablets | 2 | 7 | 2 | 0.3 |
| | 4 | 27 | 2 | 0.07 |
| | 6 | 46 | 2 | 0.04 |

*NA = Not applicable

Several of the tablets from the above tests were examined visually after exposure to SBB/SIF. Tablets with ethylcellulose coatings appeared to have absorbed fats or fat digestion products onto the surface, with the cores completely dry, or only partially damp inside. In contrast, the cores of the tablets with cellulose acetate coatings appeared to be damp to the center, with the coating remaining unchanged over the course of the experiment.

### Example 8

The ethylcellulose coated pseudoephedrine tablets of Example 3 were dosed to 36 subjects (18 male and 18 female) using an open, single dose, randomized, two-way crossover study with a wash-out period of at least seven day between doses. The tablets were administered under fasting and fed conditions. The fasted subjects were fasted for 10 hours before dosing and for 4 hours following dosing. The fed subjects were dosed 5 minutes after eating a high fat breakfast, consisting of

2 slices of white toast with butter

2 eggs fried in butter

2 slices of bacon

6 oz of hash brown potatoes

8 oz of whole milk

Blood was collected periodically to 72 hours after each dose. Samples were analyzed using HPLC methods wherein, as part of a cleanup procedure, plasma samples were treated with sodium hydroxide and an internal standard, phenylpropanolamine, added. The samples thus teated were extracted with ethyl ether, and then pseudoephedrine and internal standard were back extracted into 0.0085% aqueous phosphoric acid. The samples were then quantitated using a CN-phase analytical column (Zorbax® CN, DuPont Chromatography Products), an isocratic mobile phase consisting of 25% acetonitrile and 75% 0.0025 M potassium phosphate monobasic, with UV detection (Kratos 783 ultraviolet detector) at 208 nm. Sustained pseudoephedrine levels were seen in the fasted subjects, while low pseudoephedrine levels were seen in the fed subjects, as shown in Table VII below. The data show that for any time from 3 to 24 hours after ingestion, the concentration of pseudoephedrine in the blood for the fed subjects was less than about 0.11-fold that of the fasted subjects.

The results are further summarized in Table VIII, showing the maximum concentration in the blood (Cₘₐₓ), the time to achieve the maximum concentration in the blood (Tₘₐₓ), and the area under the concentration in the blood versus time curve (AUC) during the 48-hour test. The data show that the Cₘₐₓ and AUC for the fed subjects were only 0.06 and 0.09-fold that of the fed subjects, while Tₘₐₓ was 2.96-fold that of the fed subjects.

TABLE VII
Mean Plasma Pseudoephedrine Concentrations for Controlled
Release Tablets with Asymmetric Ethylcellulose Coatings

| Time | Fasted | Fed | Ratio |
|---|---|---|---|
| (hrs) | (ng/mL) | (ng/mL) | Fed/Fasted |
| 0 | <5 | <5 | NA |
| 1.5 | <5 | <5 | NA |
| 2 | 6.5 | <5 | <0.76 |
| 3 | 44.6 | <5 | <0.11 |
| 4 | 104 | 6.7 | 0.06 |
| 5 | 199 | 11.3 | 0.06 |
| 6 | 234 | 11.4 | 0.05 |
| 8 | 299 | 12.8 | 0.04 |
| 10 | 335 | 12.6 | 0.04 |
| 12 | 332 | 14.4 | 0.04 |
| 16 | 334 | 16.4 | 0.05 |
| 20 | 279 | 15.3 | 0.05 |
| 24 | 227 | 19.9 | 0.09 |
| 36 | 83.5 | 21.8 | 0.26 |
| 48 | 26.9 | 6.5 | 0.24 |
| 60 | 9.3 | <5 | <0.53 |
| 72 | <5 | <5 | NA |

TABLE VIII
Summary of Fed versus Fasted Pseudoephedrine Delivery for
Controlled Release Tablets with Asymmetric Ethylcellulose Coatings

| Item | Fasted | Fed | Ratio Fed/Fasted |
|---|---|---|---|
| Cₘₐₓ (ng/mL) | 364 ± 75.2 | 21.8 | 0.06 |
| Tₘₐₓ (hr) | 12.2 ± 3.3 | 36 | 2.95 |
| AUC (ng-hr/mL) | 8760 ± 1950 | 795 | 0.09 |

### Example 9

The CA-coated pseudoephedrine tablets made as described in Example 4, were tested in vivo in an open, single dose, randomized 3-way crossover study with a 7 day washout between doses. Subjects were randomized to one of two groups and received pseudoephedrine (240 mg dose) on 2 separate occasions: under fasted conditions, and under fed conditions. Sequential blood samples were collected for up to 72 hours after each dose for measurement of pseudoephedrine in blood plasma.

Pseudoephedrine in plasma was assayed using the validated HPLC/UV absorbance method described in Example 8. The assay is linear over the range of 5.00 to 500 ng/mL. Concentrations below the lower limit of quantification (5.00 ng/mL) are reported as 0.0 ng/mL in all concentration tables and taken as 0.0 ng/mL for all data analyses.

Maximum plasma pseudoephedrine concentration (Cₘₐₓ) and the time of the first occurrence of each subject's Cₘₐₓ (Tₘₐₓ) were based on direct observation of the data. Half-life (T_{1/2}) was calculated as the natural logarithm of 2 (0.6931) divided by the rate constant for elimination of drug from the blood plasma (Kₑₗ). The area under the plasma pseudoephedrine concentration-time curve from time 0 to the time of the last measurable concentration (AUC₀₋ₜ) was estimated using the linear trapezoidal method. AUC₀₋ₜ was extrapolated to infinity (AUC_{0-∞}) by the addition of Cₑₛₜ/Kₑₗ, where Cₑₛₜ is the estimated plasma concentration at time t based on regression analysis of the terminal log-linear phase. Nominal times were used for all calculations.

The pharmacokinetic parameters of pseudoephedrine for each of the treatments are given in Table XI. Mean (±SD) Cₘₐₓ values were 329±59 and 299±58 ng/mL for fasted and fed drug release, respectively. Corresponding mean Tₘₐₓ values were 11.2±1.7 and 11.2±3.2 h. Mean AUC_{0-∞} values were similar, 7120±915 and 6780±1030 ng-h/mL, as were the mean terminal T_{1/2} values, 8.4±2.1 and 7.6±1.7 h, for fasted and fed drug release, respectively. The relative bioavailability values for pseudoephedrine, comparing the drug release under fed versus fasted conditions, are given in Table XII. The average relative bioavailability of pseudoephedrine was 95±10% for the tablet administered under fed conditions versus fasted conditions. Individual plasma pseudoephedrine concentrations are provided in Tables XIII and XIV. Administration of the cellulose acetate coated tablets with food had no significant effect on the Cₘₐₓ, Tₘₐₓ, or AUC_{0-∞} of pseudoephedrine.

TABLE XI
Pharmacokinetic Parameters of Pseudoephedrine in 12 Healthy Males
after Single Dose Administration of the Cellulose Acetate Coated Tablet under
Both Fasted and Fed Conditions

| | Cₘₐₓ (ng/mL) | | | Tₘₐₓ (h) | | | T_{1/2} (h) | | | ACU_{0-∞} (ng-h/mL) | | |
|---|---|---|---|---|---|---|---|---|---|---|---|---|
| | | | Ratio | | | Ratio | | | Ratio | | | Ratio |
| Subject | Fasted | Fed | Fed/Fasted | Fasted | Fed | Fed/Fasted | Fasted | Fed | Fed/Fasted | Fasted | Fed | Fed/Fasted |
| 1 | 323 | 276 | 0.85 | 12 | 12 | 1.00 | 59 | 5.7 | 0.97 | 6670 | 5820 | 0.87 |
| 2 | 284 | 361 | 1.27 | 12 | 12 | 1.00 | 7.9 | 7 | 0.89 | 7250 | 7690 | 1.06 |
| 4 | 450 | 294 | 0.65 | 12 | 16 | 1.33 | 7.5 | 6.5 | 0.87 | 7870 | 7070 | 0.90 |
| 5 | 301 | 294 | 0.98 | 8 | 8 | 1.00 | 10.4 | 11.5 | 1.11 | 6590 | 6070 | 0.92 |
| 6 | 392 | 358 | 0.91 | 12 | 8 | 0.67 | 7.5 | 7.2 | 0.96 | 7300 | 7360 | 1.01 |
| 7 | 254 | 215 | 0.85 | 12 | 8 | 0.67 | 10.9 | 6.7 | 0.61 | 6320 | 5110 | 0.81 |
| 9 | 361 | 384 | 1.06 | 12 | 8 | 0.67 | 11.1 | 7.5 | 0.68 | 8570 | 8200 | 0.96 |
| 10 | 365 | 314 | 0.86 | 12 | 12 | 1.00 | 6.5 | 6.8 | 1.05 | 8380 | 7890 | 0.94 |
| 11 | 267 | 214 | 0.80 | 8 | 12 | 1.50 | 10.6 | 7.7 | 0.73 | 6410 | 5900 | 0.92 |
| 12 | 296 | 283 | 0.96 | 12 | 16 | 1.33 | 6.2 | 9.4 | 1.52 | 5860 | 6680 | 1.14 |
| Mean | 329 | 299 | 0.92 | 11.2 | 11.2 | 1.02 | 8.4 | 7.6 | 0.94 | 7120 | 6780 | 0.95 |
| SD | 59 | 58 | 0.16 | 1.7 | 3.2 | 0.28 | 2.1 | 1.7 | 0.25 | 915 | 1030 | 0.09 |
| % CV | 18 | 19 | 17 | 15 | 29 | 28 | 25 | 22 | 26 | 13 | 15 | 10 |

TABLE XII
Single Dose Bioavailability (%) of 240 mg Pseudoephedrine Coated
with Cellulose Acetate in 12 Healthy Males under Fed versus
Fasted Conditions

| Subject | CA (fed) vs. CA (fasted) |
|---|---|
| 1 | 87 |
| 2 | 106 |
| 4 | 90 |
| 5 | 92 |
| 6 | 101 |
| 7 | 81 |
| 9 | 96 |
| 10 | 94 |
| 11 | 92 |
| 12 | 114 |
| Mean | 95 |
| SD | 10 |
| % CV | 11 |

TABLE XIII
Plasma Pseudoephedrine Concentrations (ng/mL) in 12 Healthy
Males after Single Dose Administration under Fasted Conditions of a Cellulose
Acetate Coated Tablet Containing 240 mg of Pseudoephedrine Hydrochloride

| | | Plasma Pseudoephedrine Concentrations (ng/mL)¹ at Hour | | | | | | | | | | | |
|---|---|---|---|---|---|---|---|---|---|---|---|---|---|
| Subject | Day | 0 | 0.5 | 1 | 1.5 | 2 | 4 | 8 | 12 | 16 | 24 | 48 | 72 |
| 1 | 8 | 0.01 | 0 | 0 | 10.3 | 26.3 | 72.3 | 276 | 323 | 284 | 136 | 6.9 | 0 |
| 2 | 8 | 0 | 0 | 0 | 7.5 | 14 | 145 | 273 | 284 | 242 | 171 | 16.3 | 0 |
| 3 | 1 | 0 | 0 | 6.8 | 18.6 | 40.7 | 219 | 289 | 337 | 283 | 120 | 24.5 | 88.1 |
| 4 | 1 | 0 | 0 | 0 | 19.4 | 26.5 | 218 | 349 | 450 | 300 | 147 | 0 | 0 |
| 5 | 15 | 0 | 0 | 0 | 9.6 | 20 | 218 | 301 | 274 | 212 | 123 | 0 | 0 |
| 6 | 15 | 0 | 0 | 5.7 | 12 | 38.5 | 187 | 318 | 392 | 312 | 133 | 0 | 0 |
| 7 | 15 | 0 | 0 | 6.3 | 19.6 | 34.8 | 147 | 238 | 254 | 237 | 123 | 0 | 0 |
| 8 | 1 | 6.18 | 0 | 0 | 11.7 | 38.6 | 124 | 393 | 319 | 283 | 147 | 6 | 0 |
| 9 | 8 | 0 | 0 | 8.3 | 23.6 | 36.6 | 173 | 356 | 361 | 287 | 145 | 38.4 | 0 |
| 10 | 8 | 0 | 0 | 0 | 14.7 | 30.4 | 182 | 299 | 365 | 316 | 187 | 11.4 | 0 |
| 11 | 15 | 0 | 0 | 0 | 12.1 | 28.6 | 138 | 267 | 229 | 203 | 128 | 25.7 | 0 |
| 12 | 1 | 0 | 0 | 0 | 11.9 | 28 | 146 | 285 | 296 | 220 | 101 | 5.6 | 0 |
| Mean | | — | — | — | 14.2 | 30.3 | 164 | 304 | 324 | 265 | 138 | 16.9 | — |
| SD | | — | — | — | 4.9 | 8.1 | 44.2 | 43.6 | 62.2 | 39.9 | 23.3 | 11.8 | — |
| % CV | | — | — | — | 35 | 27 | 27 | 14 | 19 | 15 | 17 | 70 | — |

¹Concentrations <5.0 ng/mL are reported as zero

TABLE XIV
Plasma Pseudoephedrine Concentrations (ng/mL) in 12 Healthy
Males after Single Dose Administration under Fed Conditions of a Cellulose
Acetate Coated Tablet Containing 240 mg of Pseudoephedrine Hydrochloride

| | | Plasma Pseudoephedrine Concentrations (ng/mL)¹ at Hour | | | | | | | | | | | |
|---|---|---|---|---|---|---|---|---|---|---|---|---|---|
| Subject | Day | 0 | 0.5 | 1 | 1.5 | 2 | 4 | 8 | 12 | 16 | 24 | 48 | 72 |
| 1 | 15 | 0.01 | 0 | 0 | 0 | 0 | 55.8 | 243 | 276 | 268 | 116 | 5.5 | 0 |
| 2 | 1 | 0 | 0 | 0 | 0 | 0 | 85.1 | 299 | 361 | 278 | 177 | 13.1 | 0 |
| 4 | 8 | 0 | 0 | 0 | 0 | 0 | 39.3 | 278 | 274 | 294 | 172 | 10.6 | 0 |
| 5 | 8 | 0 | 0 | 0 | 0 | 0 | 129 | 294 | 239 | 212 | 111 | 0 | 0 |
| 6 | 1 | 0 | 0 | 0 | 5.6 | 27.1 | 151 | 358 | 322 | 261 | 144 | 12.7 | 0 |
| 7 | 8 | 0 | 0 | 0 | 21 | 44.5 | 82.3 | 215 | 215 | 182 | 114 | 7.4 | 0 |
| 8 | 8 | 0 | 0 | 0 | 0 | 0 | 95.1 | 338 | 265 | 295 | 141 | 9.2 | 0 |
| 9 | 1 | 0 | 6.1 | 8 | 12.5 | 41.7 | 191 | 384 | 350 | 305 | 152 | 15.9 | 0 |
| 10 | 15 | 0 | 0 | 0 | 0 | 5.9 | 153 | 241 | 314 | 298 | 196 | 12.9 | 0 |
| 11 | 1 | 0 | 0 | 0 | 0 | 17.2 | 121 | 187 | 214 | 206 | 148 | 13.1 | 0 |
| 12 | 15 | 0 | 0 | 0 | 0 | 8.9 | 68.4 | 201 | 281 | 283 | 150 | 12.4 | 5.3 |
| Mean | | — | — | — | — | 24.2 | 106 | 276 | 283 | 262 | 147 | 11.3 | — |
| SD | | — | — | — | — | 16.4 | 46.6 | 65.3 | 49.8 | 42.5 | 27.1 | 3.1 | — |
| % CV | | — | — | — | — | 68 | 44 | 24 | 18 | 16 | 18 | 27 | — |

¹Concentrations <5.0 ng/mL are reported as zero

### Example 10

Dissolution tests were performed using ethylcellulose-coated Sunepitron tablets of Example 5 using a USP II dissolution apparatus with 400 mL of a high fat (see Table XV) dissolution media at 37° C. and a 100 RPM paddle speed. The paddle height was adjusted downward 0.5 cm from the standard USP distance to provide better stirring with the smaller dissolution volume. The amount of sunepitron released at each time point was determined by an HPLC assay of the residual amount of drug in the tablet. The HPLC system used for both of these methods was a Hewlett Packard (HP) HP1050 (now owned by Agilent Technologies, Wilmington, Del.). The column was a Waters Puresil C18 Reverse Phase with 5 micron particles, column size 150×3.9 mm, part no. WAT 044345 (or equivalent). The mobile phase was a pH 4.6 buffer (0.05M ammonium acetate)/methanol/acetonitrile (91/3/6 v/v). The assay was run isocratic using a flow rate of 2 mL/minute using a UV detector set at 238 nm.

Table XVI shows the dissolution profiles for the ethylcellulose coated tablets in both the high fat media and in distilled water (50 RPM paddle speed and 900 mL). The data show that the rate of drug release from the tablets tested in the high-fat media was much slower than that of the tablets tested in distilled water. The HPLC assay for the in vitro dissolution test used a Waters Novapak C18 Reverse Phase (7.5 cm×3.9 mm) part no. 11670 column. The mobile phase was a pH 5 buffer (consisting of 0.1 % v/v triethylamine (TEA) and 0.2% v/v Glacial acetic acid)/methanol (75/25 v/v). The assay was run isocratic using a flow rate of 1 mL/minute and a UV detector set at 238 nm.

TABLE XV
High Fat Dissolution Media

2 pieces of white toast with butter
2 strips of bacon
6 oz. of hashbrowns
2 eggs scrambled in butter
8 oz. of whole milk or about 250 mL
8 g of extra butter
250 mL of SIF with enzymes (pancreatin)*
*USP SIF (Simulated Intestinal Fluid) was made as follows: 6.8 g of monobasic potassium phosphate was dissolved in 250 mL of water; 190 mL of 0.2 N sodium hydroxide was mixed with 400 mL of water and combined with the potassium phosphate solution; 10 g of pancreatin was added, and pH of the resulting solution was adjusted to 7.5 ± 0.1 with 0.2 N sodium hydroxide. Water was added for a final volume of 1000 mL.
#The high-fat dissolution media was mixed in an industrial single speed Waring Blender and enough media was made to fill 2 dissolution vessels with 40 mL of media.

TABLE XVI
Sunepitron Released From Ethylcellulose Coated Tablets in High
Fat and Low Fat Media

| | % Sunepitron Delivered (Range) | |
|---|---|---|
| Time (hours) | Water (n = 6) | High Fat (n = 3) |
| 0 | 0 | 0 |
| 1 | 6.1 (3.5-8.8) | |
| 2 | | |
| 4 | 34.6 (30.4-40.1) | |
| 6 | | 25.9 (16.0-33.3) |
| 8 | 70.6 (66.3-77.8) | |
| 12 | 91.8 (88.7-96.4) | |
| 16 | 98.6 (95.9-100) | |
| 24 | 103.0 (99.7-104) | 42.3 (27.5-66.2) |

The ethylcellulose coated tablets of Example 5 were also exposed to simulated gastric fluid with pepsin (SGF) for 1, 2 or 4 hours (900 mL, 50 rpm, 37° C.) before being transferred to the high fat dissolution media described above to approximate gastrointestinal transit. Dissolution data is presented in Table XVII. The data show that the dosage form delivered sunepitron in SGF at a rate comparable to the initial release profile in distilled water (see Table XVI). After transferring to the high fat media the rate of drug release decreases, and ultimately stops before all the drug has been delivered.

TABLE XVII
Dissolution of Sunepitron Ethylcellulose Coated Tablets in SGF,
Followed Transfer to High Fat Media (HFM)

| | | % Sunepitron | | % Sunepitron | TOTAL % |
|---|---|---|---|---|---|
| | Hours | Dissolved | Hours | Dissolved | Sunepitron |
| Tablet # | in SGF | in SGF | In HFM | in HFM | Dissolved |
| 1 | 0 | 0 | 4 | 14.87 | 14.87 |
| 2 | 0 | 0 | 8 | 13.27 | 13.27 |
| 3 | 1 | 7.17 | 4 | 20.37 | 27.54 |
| 4 | 1 | 8.49 | 5 | 16.12 | 24.61 |
| 5 | 2 | 16.19 | 6 | 10.45 | 26.64 |
| 6 | 2 | 19.56 | 8 | 11.31 | 30.86 |
| 7 | 4 | 36.70 | 4 | 23.54 | 60.23 |
| 8 | 4 | 38.88 | 8 | 25.72 | 64.60 |

The release of sunepitron from tablets coated with ethylcellulose (tablets of Example 5) or cellulose acetate (tablets of Example 6) into high fat media was compared. The data are presented in Table XVIII. The sunepitron release rate in high fat dissolution media is much faster for the cellulose acetate coated tablets than the ethylcellulose coated tablets.

TABLE XVIII
Comparison of Sunepitron Release for Ethylcellulose and
Cellulose Acetate coated Tablets in High Fat Dissolution Media

| Coating Type | Media | % released in 8 hrs | % released in 24 hrs |
|---|---|---|---|
| Ethylcellulose | High Fat | 4.0 | 42.3 |
| Cellulose acetate | High Fat | 64.6 | 94.3 |

### Example 11

The ethylcellulose-coated tablets of Example 5 were dosed to 12 subjects using an open, single dose, randomized four-way crossover study with at least three days between treatments. The tablets were administered to subjects under four conditions: (1) subjects were fasted for at least 8 hours before dosing and for 4 hours following dosing (Example 11A); (2) dosing occurred one hour prior to eating breakfast (Example 11 B), (3) dosing occurred immediately after breakfast (20 minutes after the breakfast was served) (Example 11C), and (4) dosing occurred two hours after the breakfast was consumed (Example 11D). The fed subjects ate a high fat breakfast consisting of the following:

2 pieces of toast with 2 pats of butter

2 eggs fried in butter

2 strips of bacon

6 oz of hash brown potatoes

8 oz of whole milk

Blood was collected periodically to 24 hours after each dose. Samples were analyzed using previously validated HPLC methods. The mean Cₘₐₓ and AUC values of each dosing group was divided by the values obtained for the control group (Example 11A). These results are summarized in Table XIX below and show that the Cₘₐₓ for the subjects who were dosed 1 hour before a high-fat breakfast was 0.93-fold that of the control group (Example 11A). However, when dosed 20 min or 2 hours after having a high-fat breakfast, the Cₘₐₓ of the fed subjects was only 0.57- to 0.29-fold that of the fasted subjects (Example 11A). The AUC for the fed subjects for all cases was less than 0.59-fold that of the fasted subjects.

TABLE XIX
Summary of Fed versus Fasted Sunepitron Delivery for Controlled
Release Tablets with Ethylcellulose Coatings

| | | Cₘₐₓ/(Cₘₐₓ | AUC/(AUC Example |
|---|---|---|---|
| Example | Dosing Method | Example 11A) | 11A) |
| 11B | 1 hour before | 0.93 | 0.59 |
| | high-fat breakfast | | |
| 11C | 20 min after high- | 0.57 | 0.16 |
| | fat breakfast | | |
| 11D | 2 hr after high-fat | 0.29 | 0.11 |
| | breakfast | | |

### Example 12

Multiple 10 mg cellulose-acetate-coated controlled-release sunepitron tablets of Example 6, resulting in dose of either 30 mg or 60 mg, were given to 12 male subjects using a randomized double-blind, placebo-controlled two-way crossover study with a one week wash-out period between doses. The tablets were administered under fasted and fed conditions. The fasted subjects were fasted for 8 hours before dosing and for 4 hours following dosing. The fed subjects were dosed 10 minutes after eating a high fat breakfast consisting of the following:

2 slices of white toast with butter and jelly

2 eggs

bacon and ham

8 oz of whole milk

Blood was collected periodically to 48 hours after each dose. Samples were analyzed using previously validated HPLC methods. The mean Cₘₐₓ and AUC of each dosing group are summarized in Table XX below, and show that for both the 30-mg and 60-mg dose, the Cₘₐₓ and AUC for the fed subjects were 0.97- to 1.08-fold that of the fasted subjects.

TABLE XX
Summary of Fed versus Fasted Sunepitron Delivery for Controlled
Release Tablets with Asymmetric Cellulose Acetate Coatings

| | | | | |
|---|---|---|---|---|
| | | | | Ratio |
| Dose | Parameter | Fasted | Fed | Fed/Fasted |

| | | | | | |
|---|---|---|---|---|---|
| 30 mg | Cₘₐₓ | (ng/mL) | 2.73 | 2.96 | 1.08 |
| 30 mg | AUC | (ng-h/mL) | 31 | 30 | 0.97 |
| 60 mg | Cₘₐₓ | (ng/mL) | 3.51 | 3.79 | 1.08 |
| 60 mg | AUC | (ng-h/mL) | 39 | 41 | 1.05 |

The pseudoephedrine-containing tablets of Example 3 were dissolution tested as follows. A 100 ml sample of 5 wt % of 50% hydrolyzed model oil (37 wt % olive oil, 15 wt % Myverol® 18-99, 23 wt % oleic acid, 9 wt % tripalmitin, 4 wt % Imwitor 191®, 5 wt % palmitic acid, 3 wt % tributyrin, 2 wt % butyric acid, 1 wt % monobutyrin, and 1 wt % lecithin) in a simulated intestinal buffer containing no enzymes (SIN, 0.05 M KH₂PO₄ adjusted to pH 6.8 with 0.2 N NaOH) was placed in a screw-top Nalgene® container affixed to a vertical rotating wheel in a 37° C. temperature-controlled chamber. Two tablets of Example 3 were added to the container and the wheel rotated for 6 hours.

After 6 hours, the tablets were removed from the container and cut open. The fraction of the core that had wet with the dissolution media was estimated. The amount of pseudoephedrine remaining in the tablets after 6 hours was determined by residual analysis using the techniques described in Example 7. The amount of pseudoephedrine released after 6 hours was calculated by subtracting the amount of pseudoephedrine remaining in the tablet from the total pseudoephedrine initially present in the tablet. Similar tests were performed using a dissolution solution of SIN but containing no 50% hydrolyzed model oil. The results of these tests are shown in Table XXI.

TABLE XXI
Appearance and Drug Release from Pseudoephedrine Tablets

| | | Core | Pseudoephedrine |
|---|---|---|---|
| | | Wetting | Released |
| Dissolution | Coating | (% wet | at 6 hours |
| Media | Appearance* | at 6 hr) | (%) |
| SIN (no 50% | No change in | 60 | 32 and 40 |
| hydrolyzed | appearance, | | |
| model oil) | intact. | | |
| SIN (with 50% | Slimy | 0 | 7 and 10 |
| hydrolyzed | | | |
| model oil) | | | |

*Key to observations. Slimy: coating slick to touch and beginning to dissolve.

The data in Table XXI show that when tested in SIN without the 50% hydrolyzed oil, about 60% of the core of the tablets of Example 3 had become wet within 6 hours. In addition, 32% and 40% of the pseudoephedrine had released from the two tablets tested. However, after testing for 6 hours in SIN with the 50% hydrolyzed oil, the tablet coating was slick to the touch and appeared to be beginning to dissolve. In addition, the tablet cores had not wet and only 7% and 10% of the pseudoephedrine had released from the two tablets tested. These data demonstrate that the ethyl cellulose coating used in the tablets of Example 3 is not suitable for use in this invention. In addition, this example demonstrates that 50% hydrolyzed oil can be used as an in vitro test to identify coatings that are susceptable to changes in performance due to fats and digestion products of fats in vivo.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A method for the controlled-release of an active substance into a use environment, comprising:
a. preparing a controlled-release delivery composition comprising an active-substance-containing core and an asymmetric polymeric coating thereon, wherein the polymer used to form said asymmetric polymeric coating is one which, when tested by soaking for at least 16 hours in an aqueous solution comprising 0.5 wt % dietary fat, gains less than about 15 wt %; and
b. administering said composition to said use environment,
said use environment comprising at least about 0.5 wt % of dietary fat.

2. A method for the controlled-release of an active substance into a use environment, comprising:
a. preparing a controlled-release delivery composition comprising an active-substance-containing core and an asymmetric polymeric coating thereon, wherein the time to release 50% of said active substance from said composition into said use environment is at least 0.5-fold, but less than 2.0-fold the time required for said composition to release 50% of said active substance into a control use environment comprising less than about 0.1% of dietary fat, and
b. administering said composition to said use environment, said use environment comprising at least about 0.5 wt % of dietary fat.

3. A method for the controlled-release of an active substance into a use environment, comprising:
a. preparing a controlled-release delivery composition comprising an active-substance-containing core and an asymmetric polymeric coating thereon, wherein the amount of drug released from said composition at any time between the 2^{nd} and 10^{th} hour following introduction of said composition to said use environment is at least 0.5-fold, but less than 2.0-fold the amount of said drug released at the same time between the 2^{nd} and 10^{th} hour by said composition into a control use environment comprising less than about 0.1% of dietary fat, and
b. administering said composition to said use environment,
said use environment comprising at least about 0.5 wt % of dietary fat.

4. A method for the controlled-release of an active substance into a use environment, comprising:
a. preparing a controlled-release delivery composition comprising an active-substance-containing core and an asymmetric polymeric coating thereon, wherein the average rate of drug release from said composition between the 2^{nd} and 10^{th} hour after introduction into said use environment is at least 0.5-fold, but less than 2.0-fold the average rate of drug release provided by said composition in a control use environment comprising less than about 0.1% of dietary fat, and
b. administering said composition to said use environment,
said use environment comprising at least about 0.5 wt % of dietary fat.

5. A method for the controlled-release of an active substance into a use environment, comprising:
a. preparing a controlled-release delivery composition comprising an active-substance-containing core and an asymmetric polymeric coating thereon, wherein the composition provides a maximum concentration of said active substance in said use environment that is at least 0.5-fold, but less than 2.0-fold the maximum concentration provided by said composition in a control use environment comprising less than about 0.1% of dietary fat, and
b. administering said composition to said use environment,
said use environment comprising at least about 0.5 wt % of dietary fat.

6. A method for the controlled-release of an active substance into a use environment, comprising:
a. preparing a controlled-release delivery composition comprising an active-substance-containing core and an asymmetric polymeric coating thereon, wherein the composition provides an area under the active substance concentration versus time curve (AUC) for any period of at least 90 minutes between the time of introduction to said use environment and about 270 minutes following introduction to said use environment that is at least 0.5-fold, but less than 2.0-fold the AUC provided by said composition in a control use environment comprising less than about 0.1% of dietary fat, and
b. administering said composition to said use environment,
said use environment comprising at least about 0.5 wt % of dietary fat.

7. A method for the controlled-release of an active substance into a use environment, comprising:
a. preparing a controlled-release delivery composition comprising an active-substance-containing core and an asymmetric polymeric coating thereon, wherein the composition provides a relative bioavailability in said use environment that is at least 0.5-fold, but less than 2.0-fold the relative bioavailability provided by said composition in a control use environment comprising less than about 0.1 % of dietary fat, and
b. administering said composition to said use environment,
said use environment comprising at least about 0.5 wt % of dietary fat.

8. A therapeutic package, comprising:
a container,
a controlled-release delivery composition for the controlled release of an active substance into a use environment, comprising an active-substance-containing core and an asymmetric polymeric coating thereon, wherein said delivery composition satisfies any one or more of the following conditions (i) through (vii):
(i) the polymer used to form said polymeric coating is one which, when tested by soaking for at least 16 hours in an aqueous solution comprising 0.5 wt % dietary fat, gains less than about 15 wt %;
(ii) the time to release 50% of said active substance from said composition into said use environment is at least 0.5-fold, but less than 2.0-fold the time required for said composition to release 50% of said active substance into a control use environment comprising less than about 0.1 % of dietary fat;
(iii) the amount of drug released from said composition at any time between the 2^{nd} and 10^{th} hour following introduction of said composition to said use environment is at least 0.5-fold, but less than 2.0-fold the amount of said drug released at the same time between the 2^{nd} and 10^{th} hour by said composition into a control use environment comprising less than about 0.1% of dietary fat;
(iv) the average rate of drug release from said composition between the 2^{nd} and 10^{th} hour after introduction into said use environment is at least 0.5-fold, but less than 2.0-fold the average rate of drug release provided by said composition in a control use environment comprising less than about 0.1% of dietary fat;
(v) the composition provides a maximum concentration of said active substance in said use environment that is at least 0.5-fold, but less than 2.0-fold the maximum concentration provided by said composition in a control use environment comprising less than about 0.1% of dietary fat;
(vi) the composition provides an area under the active substance concentration versus time curve (AUC) for any period of at least 90 minutes between the time of introduction to said use environment and about 270 minutes following introduction to said use environment that is at least 0.5-fold, but less than 2.0-fold the AUC provided by said composition in a control use environment comprising less than about 0.1% of dietary fat; or
(vii) the composition provides a relative bioavailability in said use environment that is at least 0.5-fold, but less than 2.0-fold the relative bioavailability provided by said composition in a control use environment comprising less than about 0.1% of dietary fat,
and, associated with said package, written matter non-limited as to whether the dosage form can be taken with or without food.

9. A method as claimed in any one of claims 1-7, or a therapeutic package as claimed in claim 8, wherein said controlled-release delivery composition is embodied as an osmotic dosage form.

10. A method or therapeutic package as claimed in claim 9, wherein said osmotic dosage form comprises a homogeneous core, a bursting osmotic core, or a bursting coated swelling core.

11. A method as claimed in any one of claims 1-7, or a therapeutic package as claimed in claim 8, wherein said controlled-release delivery composition is embodied as a hydrogel-driven device.

12. A method as claimed in any one of claims 1-7, or a therapeutic package as claimed in claim 8, wherein said controlled-release delivery composition is embodied as a diffusion device.

13. A method as claimed in any one of claims 1-7, or a therapeutic package as claimed in claim 8, wherein said active substance is selected from antihypertensives, antianxiety agents, anticlotting agents, anticonvulsants, blood glucose-lowering agents, decongestants, antihistamines, antitussives, antineoplastics, beta blockers, antiinflammatories, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, and cholesterol ester transfer protein inhibitors.

14. A method as claimed in any one of claims 1-7, or a therapeutic package as claimed in claim 8, wherein said active substance is selected from prazosin, nifedipine, amlodipine besylate, trimazosin, doxazosin, glipizide, chlorpropamide, sildenafil, sildenafil citrate, chlorambucil, lomustine, echinomycin, tubulazole, atorvastatin calcium, hydroxyzine hydrochloride, doxepin hydrochloride, betamethasone, prednisolone, aspirin, piroxicam, valdecoxib, carprofen, celecoxib, flurbiprofen, (+)-N-{4-[3-(4-fluorophenoxy)phenoxy]-2-cyclopenten-1-yl}-N-hyroxyurea, phenobarbital, acyclovir, nelfinavir, virazole, retinol, vitamin E, timolol, nadolol, apomorphine, chlorthalidone, spironolactone, dicumarol, digoxin, digitoxin, 17-methyltestosterone, testosterone, desoxycorticosterone, alfaxalone, fluoxymesterone, methanstenolone, sulpiride, [3,6-dimethyl-2-(2,4,6-trimethyl-phenoxy)-pyridin-4-yl]-(1-ethylpropyl)-amine, 3,5-dimethyl-4-(3′-pentoxy)-2-(2′,4′,6′-trimethylphenoxy)pyridine, pyroxidine, fluoxetine, paroxetine, venlafaxine, sertraline, carbenicillin indanylsodium, bacampicillin hydrochloride, troleandomycin, doxycyline hyclate, ampicillin, penicillin G, benzalkonium chloride, chlorhexidine, nitroglycerin, mioflazine, etomidate, acetazolamide, chlorzolamide, econazole, terconazole, fluconazole, voriconazole, griseofulvin, metronidazole, thiabendazole, oxfendazole, morantel, astemizole, levocabastine, cetirizine, loratadine, decarboethoxyloratadine, cinnarizine, ziprasidone, olanzepine, thiothixene hydrochloride, fluspirilene, risperidone, penfluridole, loperamide, cisapride, ketanserin, mianserin, lidocaine, acetohexamide, dimenhydrinate, cotrimoxazole, L-DOPA, THA, donepezil, famotidine, chlordiazepoxide, triazolam, alprostadil, prostacyclin, enalaprilic acid, lisinopril, oxytetracycline, minocycline, erythromycin, clarithromycin, spiramycin, azithromycin, [R—(R*S*)]-5-chloro-N-[2-hydroxy-3-{methoxymethylamino}-3-oxo-1-(phenylmethyl)propyl-1H-indole-2-carboxamide, 5-chloro-1H-indole-2-carboxylic acid [(1S)-benzyl-(2R)-hydroxy-3-((3R,4S)-dihydroxypyrrolidin-1-yl-)-3-oxypropyl]amide, [2R,4S]-4-[3,5-bis-trifluoromethyl-benzyl)-methoxycarbonyl-amino]-2-ethyl-6-trifluoromethyl-3,4-dihydro-2H-quinoline-1-carboxylic acid ethyl ester, and [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester.

15. A method as claimed in any one of claims 1-7, or a therapeutic package as claimed in claim 8, wherein said use environment is in vitro.

16. A method as claimed in any one of claims 1-7, or a therapeutic package as claimed in claim 8, wherein said use environment is in vivo.

17. A method as claimed in any one of claims 1-7, or a therapeutic package as claimed in claim 8, wherein said use environment is the human gastrointestinal tract.

18. A method as claimed in any one of claims 1-7, or a therapeutic package as claimed in claim 8, wherein said use environment contains at least 2.0 wt % of dietary fat.

19. A method as claimed in any one of claims 1-7, or a therapeutic package as claimed in claim 8, further comprising a taste making coating surrounding said polymeric coating.

20. A method as claimed in any one of claims 1-7, or a therapeutic package as claimed in claim 8, further comprising an immediate release coating of said active substance surrounding said polymeric coating.

21. A method as claimed in any one of claims 1-7, or a therapeutic package as claimed in claim 8, wherein said asymmetric polymeric coating comprises cellulose acetate, cellulose acetate butyrate, cellulose acetate proprionate, cellulose acetate phthalate, hydroxypropyl methyl cellulose acetate succinate, polymethacrylate, and mixtures and blends thereof.

22. A method as claimed in any one of claims 1-7, or a therapeutic package as claimed in claim 8, wherein said asymmetric polymeric coating comprises cellulose acetate.
